(19) United States
(12) Patent Application Publication
(10) Pub. No.: US 2016/0213232 A1
(43) Pub. Date: Jul. 28, 2016

(54) GUIDANCE DEVICE AND CAPSULE MEDICAL DEVICE GUIDANCE SYSTEM

(71) Applicant: OLYMPUS CORPORATION, Tokyo (JP)

(72) Inventors: Miho KATAYAMA, Yokohama-shi (JP); Hironao KAWANO, Tokyo (JP); Hironobu TAKIZAWA, Tokyo (JP); Shinsuke TANAKA, Tokyo (JP)

(73) Assignee: OLYMPUS CORPORATION, Tokyo (JP)

(21) Appl. No.: 15/088,348

(22) Filed: Apr. 1, 2016

Related U.S. Application Data
(63) Continuation of application No. PCT/JP2015/055059, filed on Feb. 23, 2015.

(30) Foreign Application Priority Data
Mar. 20, 2014 (JP) 2014-059224

(51) Int. Cl. A61B 1/00; A61B 1/04

(52) U.S. Cl. CPC A61B 1/00158; A61B 1/041

(57) ABSTRACT

A guidance device includes: a permanent magnet; an accommodating portion; a shielding member configured to shield the magnetic field generated by the permanent magnet; a supporting mechanism configured to support the permanent magnet between a first position and a second position; a driving unit configured to operate the supporting mechanism upon receiving an electric power supply; and a shielding member moving mechanism configured to move the shielding member between a shielding position and a non-shielding position in conjunction with an operation of the supporting mechanism causing the permanent magnet to move in a vertical direction. When the electric power supply to the driving unit stops, the shielding member moving mechanism moves the shielding member to the shielding position in conjunction with an operation of the supporting mechanism causing the permanent magnet to move to the first position.

### CROSS-REFERENCE TO RELATED APPLICATIONS

This application is a continuation of PCT international application Ser. No. PCT/JP2015/055059 filed on Feb. 23, 2015 which designates the United States, incorporated herein by reference, and which claims the benefit of priority from Japanese Patent Application No. 2014-059224, filed on Mar. 20, 2014, incorporated herein by reference.

### BACKGROUND

1. Technical Field

The disclosure relates to a guidance device and a capsule medical device guidance system for guiding, inside a subject, a capsule medical device that is introduced into the subject to perform examinations, treatment, and the like.

2. Related Art

Conventionally, a capsule medical device which is introduced into a subject to perform examinations, treatment, and the like has been developed. An example of the capsule medical device is a capsule endoscope which is formed in such a size that can be introduced into the gastrointestinal tract of a subject. The capsule endoscope is a device having an imaging function and a wireless-communication function inside a capsule-shaped casing. The capsule endoscope acquires image data by capturing the image of the inside of the organ of a subject while moving through the gastrointestinal tract by the peristaltic movement or the like after being swallowed through the mouth of the subject and wirelessly transmits the image data to a receiving device provided outside the subject. The image data received by the receiving device is taken into an image display device and is subjected to predetermined image processing. In this way, the images of the inside of the subject can be displayed on a display. A user such as a physician or a medical engineer can observe the state of the organs of the subject through the images displayed on the image display device.

In recent years, a guidance system that guides a capsule endoscope introduced into a subject with the aid of a magnetic force (this guiding is referred to as magnetic guidance) has been proposed (see Japanese Patent Application National Publication (Laid-Open) No. 2008-503310 A). In general, in such a guidance system, a permanent magnet (hereinafter also referred to as an internal permanent magnet) is provided inside the capsule endoscope, and a magnetic attracting force generated by a magnetic field generation source such as an electromagnet or a permanent magnet provided outside the capsule endoscope acts on the internal permanent magnet. In this way, the capsule endoscope introduced into the subject is magnetically guided.

However, when a permanent magnet is used as a magnetic field generation source, it is necessary to shield the magnetic field when the guidance of the capsule endoscope is not performed. This is because the magnetic field generation source used for guidance of the capsule endoscope has a very strong magnetic force and has a large influence on the surroundings. For example, WO 2007/083708 A discloses a technique of suppressing the leakage of a magnetic field to the outside or shielding the magnetic field by covering the permanent magnet with a lid portion formed of a ferromagnetic substance or accommodating the permanent magnet in a box formed of a ferromagnetic substance when the magnetic field generation source is not used (when the capsule endoscope is not guided).

### SUMMARY

In some embodiments, a guidance device is a guidance device for guiding a capsule medical device having a first permanent magnet included therein by allowing a magnetic field to act on the first permanent magnet. The guidance device includes: a permanent magnet provided outside the capsule medical device, the permanent magnet generating a magnetic field that acts on the magnet; an accommodating portion configured to accommodate the permanent magnet; a shielding member configured to shield the magnetic field generated by the permanent magnet accommodated in the accommodating portion; a supporting mechanism configured to support the permanent magnet to be movable in a vertical direction, the supporting mechanism being configured to support the permanent magnet between a first position at which the permanent magnet is accommodated in the accommodating portion and a second position at which the capsule medical device can be guided by the magnetic field generated by the permanent magnet; a driving unit configured to operate the supporting mechanism to move the permanent magnet along a vertical direction upon receiving an electric power supply; and a shielding member moving mechanism configured to move the shielding member between a shielding position at which the magnetic field generated by the permanent magnet is confined in the accommodating portion and a non-shielding position at which the magnetic field generated by the permanent magnet is not confined in the accommodating portion in conjunction with an operation of the supporting mechanism causing the permanent magnet to move in the vertical direction. When the electric power supply to the driving unit stops, the shielding member moving mechanism-moves the shielding member to the shielding position in conjunction with an operation of the supporting mechanism causing the permanent magnet to move to the first position.

In some embodiments, a capsule medical device guidance system includes: the guidance device; and the capsule medical device.

The above and other features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1A is a schematic diagram illustrating a configuration example (the non-shielding state) of a capsule medical device guidance system according to a first embodiment of the present invention;

FIG. 1B is a schematic diagram illustrating a configuration example (the shielding state) of the capsule medical device guidance system according to the first embodiment of the present invention;

FIG. 2 is a partial cross-sectional view illustrating an example of an inner structure of a capsule endoscope illustrated in FIG. 1;

FIG. 3A is a perspective view (the non-shielding state) schematically illustrating an inner configuration of a permanent magnet and an accommodating portion illustrated in FIG. 1;

FIG. 3B is a perspective view (the shielding state) schematically illustrating an inner configuration of a permanent magnet and an accommodating portion illustrated in FIG. 1;

FIG. 4A is a schematic diagram for describing a structure of a lid portion illustrated in FIG. 3A;

FIG. 4B is a schematic diagram for describing a structure of a lid portion illustrated in FIG. 3A;

FIG. 5A is a perspective view for describing an operation of a magnet holding mechanism illustrated in FIG. 3A;

FIG. 5B is a perspective view for describing an operation of a magnet holding mechanism illustrated in FIG. 3A;

FIG. 5C is a perspective view for describing an operation of a magnet holding mechanism illustrated in FIG. 3A;

FIG. 6 is a flowchart illustrating an operation of a guidance device illustrated in FIG. 1;

FIG. 7A is a schematic diagram illustrating a configuration (the non-shielding state) of an accommodating portion included in a guidance device according to Modified Example 1-1 of the first embodiment of the present invention;

FIG. 7B is a schematic diagram illustrating a configuration (the shielding state) of an accommodating portion included in a guidance device according to Modified Example 1-1 of the first embodiment of the present invention;

FIG. 8A is a schematic diagram illustrating a configuration (the non-shielding state) of an accommodating portion included in a guidance device according to Modified Example 1-2 of the first embodiment of the present invention;

FIG. 8B is a schematic diagram illustrating a configuration (the shielding state) of an accommodating portion included in a guidance device according to Modified Example 1-2 of the first embodiment of the present invention;

FIG. 9A is a schematic diagram illustrating a configuration (the non-shielding state) of a guidance device according to a second embodiment of the present invention;

FIG. 9B is a schematic diagram illustrating a configuration (the shielding state) of a guidance device according to the second embodiment of the present invention;

FIG. 10A is a perspective view illustrating a magnet holding mechanism illustrated in FIG. 9A at an enlarged scale;

FIG. 10B is a perspective view illustrating a magnet holding mechanism illustrated in FIG. 9B at an enlarged scale;

FIG. 11 is a schematic diagram illustrating a configuration of a guidance device according to Modified Example 2-1 of the second embodiment of the present invention;

FIG. 12 is a schematic diagram illustrating a configuration of a guidance device according to Modified Example 2-2 of the second embodiment of the present invention;

FIG. 13A is a schematic diagram illustrating a configuration of a guidance device according to Modified Example 2-3 of the second embodiment of the present invention;

FIG. 13B is a partial enlarged view of a stopper illustrated in FIG. 13A;

FIG. 14 is a schematic diagram illustrating a configuration of an accommodating portion of a permanent magnet used in a guidance device according to a third embodiment of the present invention;

FIG. 15 is a schematic diagram illustrating a configuration of a maintenance accommodating portion used in a guidance device according to Modified Example 3-1 of the third embodiment of the present invention;

FIG. 16 is a schematic diagram illustrating a configuration of a guidance device according to a fourth embodiment of the present invention;

FIG. 17A is a schematic diagram illustrating a magnetic field display portion (the shielding state) illustrated in FIG. 16 at an enlarged scale;

FIG. 17B is a schematic diagram illustrating a magnetic field display portion (the non-shielding state) illustrated in FIG. 16 at an enlarged scale;

FIG. 18A is a schematic diagram illustrating a magnetic field display portion (the shielding state) included in a guidance device according to Modified Example 4-1 of the fourth embodiment of the present invention;

FIG. 18B is a schematic diagram illustrating a magnetic field display portion (the non-shielding state) included in a guidance device according to Modified Example 4-1 of the fourth embodiment of the present invention;

FIG. 19A is a schematic diagram illustrating a magnetic field display portion (the shielding state) included in a guidance device according to Modified Example 4-2 of the fourth embodiment of the present invention;

FIG. 19B is a schematic diagram illustrating a magnetic field display portion (the non-shielding state) included in a guidance device according to Modified Example 4-2 of the fourth embodiment of the present invention;

FIG. 20A is a schematic diagram illustrating a magnetic field display portion (the shielding state) included in a guidance device according to Modified Example 4-3 of the fourth embodiment of the present invention;

FIG. 20B is a schematic diagram illustrating a magnetic field display portion (the non-shielding state) included in a guidance device according to Modified Example 4-3 of the fourth embodiment of the present invention;

FIG. 21A is a schematic diagram illustrating an inner configuration of a magnetic field display portion illustrated in FIG. 20A;

FIG. 21B is a schematic diagram illustrating an inner configuration of a magnetic field display portion illustrated in FIG. 20B;

FIG. 22A is a schematic diagram illustrating an appearance of a magnetic field display portion (the shielding state) included in a guidance device according to Modified Example 4-4 of the fourth embodiment of the present invention;

FIG. 22B is a schematic diagram illustrating an appearance of a magnetic field display portion (the non-shielding state) included in a guidance device according to Modified Example 4-4 of the fourth embodiment of the present invention;

FIG. 23A is a schematic diagram illustrating a configuration of a disk illustrated in FIG. 22A; and

FIG. 23B is a schematic diagram illustrating a configuration of a disk illustrated in FIG. 22B.

### DETAILED DESCRIPTION

A guidance device and a capsule medical device guidance system according to embodiments of the present invention will be described below with reference to the drawings. In the following description, although a capsule endoscope that is orally introduced into a subject and captures an image of the inside (the lumen) of the subject is illustrated as a form of a capsule medical device, the present invention is not limited to the embodiment. That is, for example, the present invention can be applied to various medical devices which are used by being inserted into a subject, such as a capsule medical device that delivers medicine or the like into a subject or a capsule medical device having a PH sensor that measures the PH inside a subject in addition to a capsule endoscope that moves in the lumen from the esophagus to the anus of a subject.

In the following description, the shape, the size, and the positional relationship in the respective drawings are schematically illustrated to such an extent that facilitates understanding of the content of the present invention. Therefore, the present invention is not limited to the shape, the size, and the positional relationship illustrated in each drawing. The same reference signs are used to designate the same parts throughout the drawings.

### First Embodiment

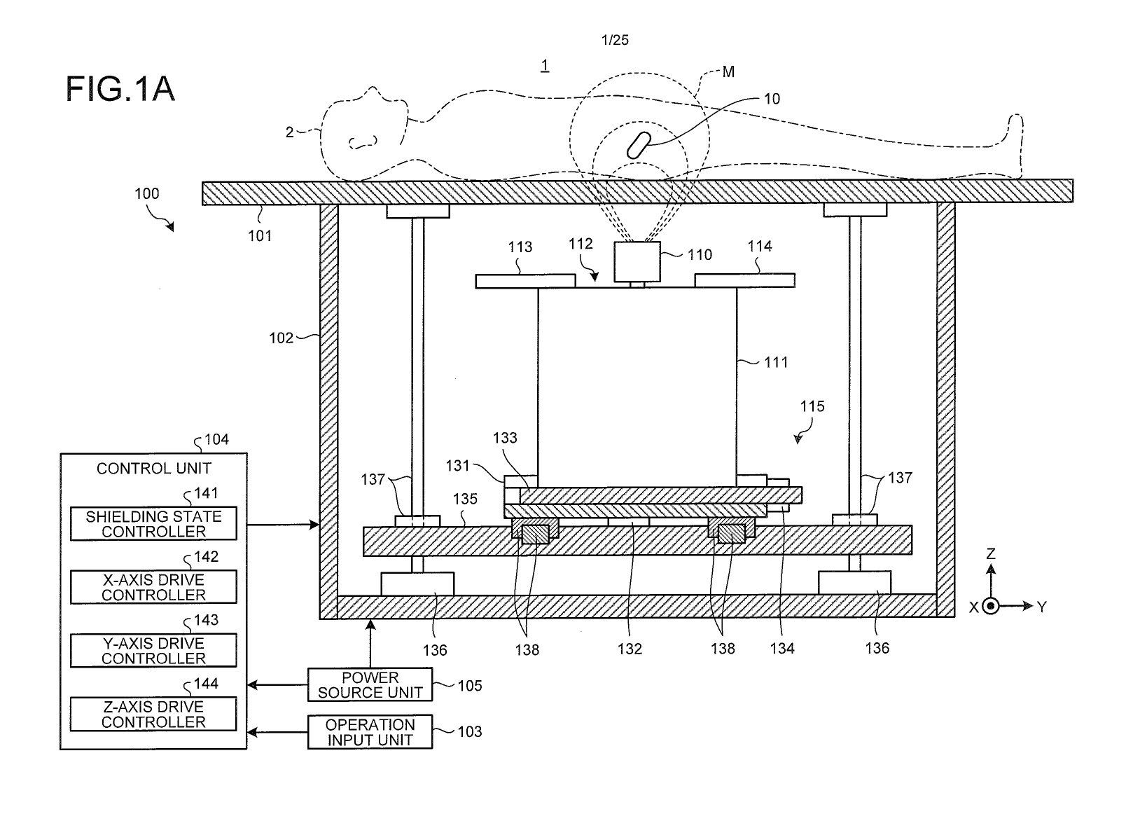

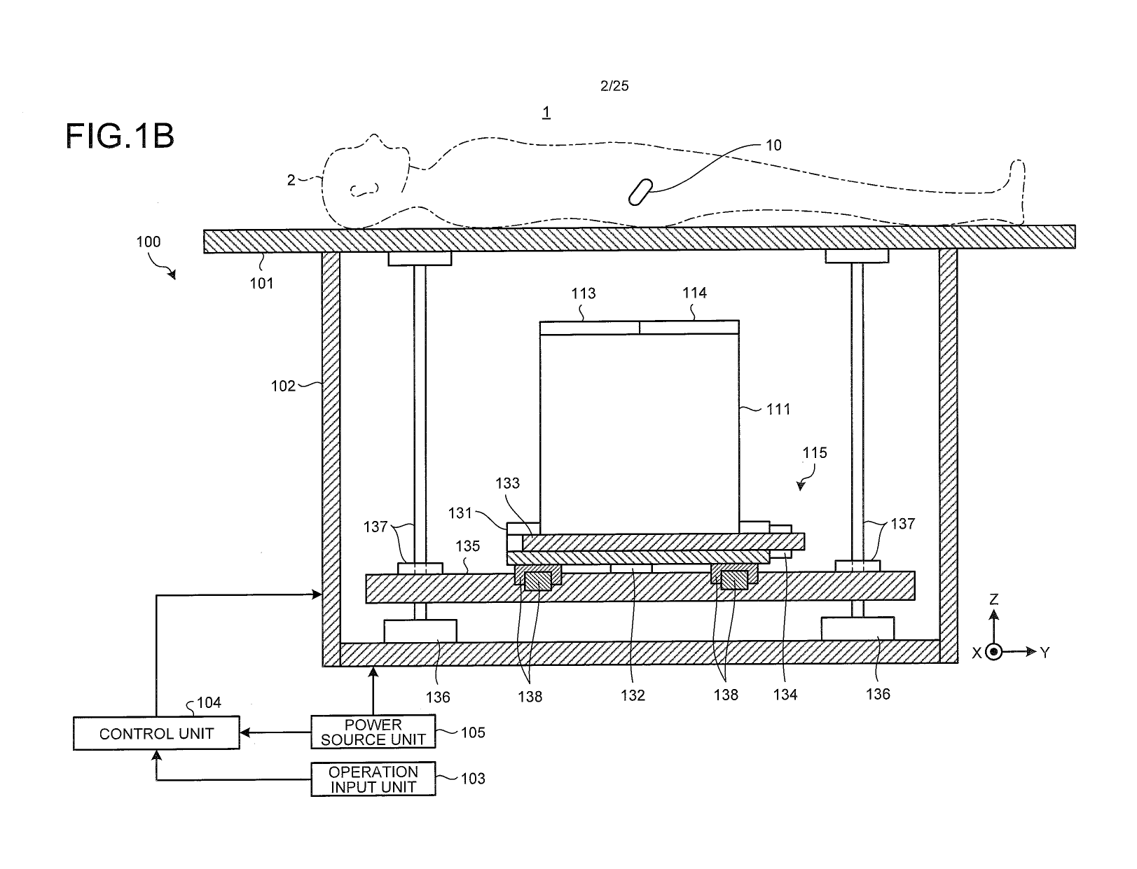

FIGS. 1A and 1B are schematic diagrams illustrating a configuration example of a capsule medical device guidance system according to the first embodiment of the present invention. As illustrated in FIGS. 1A and 1B, a capsule medical device guidance system 1 of the first embodiment includes a capsule endoscope 10 in which a permanent magnet is provided and which is used by being inserted into the body cavity of a subject 2 and a guidance device 100 that generates a magnetic field M in an area on which the subject 2 is mounted so as to act on the permanent magnet in the capsule endoscope 10 to magnetically guide the capsule endoscope 10 in the subject 2. FIG. 1A illustrates a state in which the magnetic field M is generated in the area on which the subject 2 is mounted and FIG. 1B illustrates a state in which the magnetic field M is not generated in the area. In FIG. 1B, an inner configuration of a control unit 104 is not depicted.

The capsule endoscope 10 moves through the gastrointestinal tract after being introduced into the subject 2 via oral ingestion or the like and is finally discharged outside the subject 2. In this period, the capsule endoscope 10 captures the images of the inside of the gastrointestinal tract at a predetermined cycle while being magnetically guided by the magnetic field M and wirelessly transmits the image information (image data) acquired by the capturing sequentially.

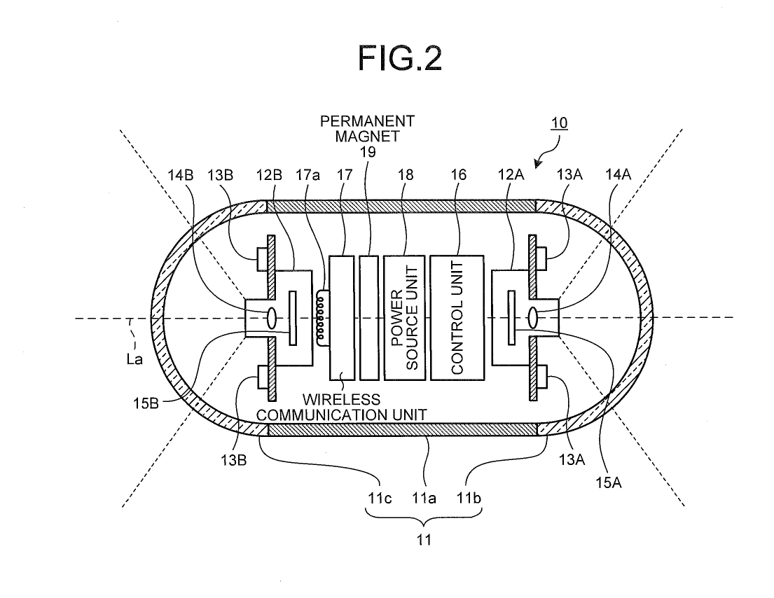

FIG. 2 is a partial cross-sectional view illustrating a configuration of the capsule endoscope 10. As illustrated in FIG. 2, the capsule endoscope 10 includes a capsule-shaped casing 11 formed in such a size that can be easily introduced into the organ of the subject 2 and imaging units 12A and 12B that capture images from different directions. Moreover, the capsule endoscope 10 includes a control unit 16 that controls respective constituent elements of the capsule endoscope 10, a wireless communication unit 17 that wirelessly transmits the image data acquired when the imaging units 12A and 12B perform capturing to the outside, and a power source unit 18 that supplies electric power to the respective constituent elements of the capsule endoscope 10. Further, the capsule endoscope 10 includes a permanent magnet 19 for enabling the guidance device 100 to perform magnetic guidance.

The capsule-shaped casing 11 is an outer casing formed in such a size that can be introduced into the organ of the subject 2 and includes a tubular casing 11a having a cylindrical shape and dome-shaped casings 11b and 11c having a dome shape. The capsule-shaped casing 11 is formed by closing both opening ends of the tubular casing 11a with the dome-shaped casings 11b and 11c. The dome-shaped casings 11b and 11c are dome-shaped optical members that are transparent to light in a predetermined wavelength range such as visible light. Moreover, the tubular casing 11a is a color casing that is substantially opaque to visible light. Such a capsule-shaped casing 11 liquid-tightly contains the imaging units 12A and 12B, the control unit 16, the wireless communication unit 17, the power source unit 18, and the permanent magnet 19.

The imaging unit 12A includes an illumination unit 13A such as an LED, an optical system 14A such as a condenser lens, and an image sensor 15A such as a CMOS image sensor or a CCD. The illumination unit 13A emits white light and illuminates an imaging visual field of the image sensor 15A over the dome-shaped casing 11b. The optical system 14A condenses light reflected from the imaging visual field and forms a subject image in the imaging visual field on an imaging surface of the image sensor 15A. The image sensor 15A acquires the image information of the subject 2 in the imaging visual field by photoelectrically converting an optical signal of the subject image formed on the imaging surface.

The imaging unit 12B includes an illumination unit 13B such as an LED, an optical system 14B such as a condenser lens, and an image sensor 15B and performs imaging over the dome-shaped casing 11c similarly to the imaging unit 12A.

The control unit 16 controls the respective operations of the imaging units 12A and 12B and the wireless communication unit 17 and controls the input and output of signals between these respective constituent elements. Moreover, the control unit 16 generates image data by applying predetermined image processing to the image information acquired by the image sensors 15A and 15B. Further, the control unit 16 causes the wireless communication unit 17 to wirelessly transmit the generated image data sequentially to the outside.

The wireless communication unit 17 includes an antenna 17a, performs a modulation process or the like on the image data acquired from the control unit 16, superimposes the image data on a radio signal, and wirelessly transmits the radio signal sequentially to the outside through the antenna 17a.

The power source unit 18 includes an electric storage unit such as a button battery or a capacitor and a switch unit such as a magnetic switch or an optical switch. The power source unit 18 switches the ON/OFF state of the power source according to light or a magnetic field applied from the outside. In the ON state, the power source unit 18 appropriately supplies the electric power of the battery or the electric storage unit to the respective constituent elements (the imaging units 12A and 12B, the wireless communication unit 17, and the control unit 16) of the capsule endoscope 10. In the OFF state, the power source unit 18 stops the electric power supply to the respective constituent elements of the capsule endoscope 10.

The permanent magnet 19 is provided to enable the magnetic guidance of the capsule endoscope 10 according to the magnetic field M generated by the guidance device 100 and is fixedly arranged inside the capsule-shaped casing 11 so that the magnetization direction thereof is inclined with respect to a long axis La. Specifically, the permanent magnet 19 is disposed so that the magnetization direction is orthogonal to the long axis La. The permanent magnet 19 moves following a change in the magnetic field M whereby the magnetic guidance of the capsule endoscope 10 by the guidance device 100 is realized.

Next, a configuration of the guidance device 100 will be described. As illustrated in FIGS. 1A and 1B, the guidance device 100 includes a bed 101 on which the subject 2 is mounted, a leg portion 102 which is a casing that supports the bed 101 and in which various devices are included, an operation input unit 103 that a user such as a physician or a medical engineer uses to operate the guidance device 100, the control unit 104 that controls the respective units of the guidance device 100 based on a signal input based on an operation on the operation input unit 103, and a power source unit 105 that supplies electric power to the respective units of the guidance device 100. Among these constituent elements, the bed 101 is supported by the leg portion 102 so that a mounting surface of the subject 2 is horizontal. In the following description, the mounting surface (horizontal surface) of the subject 2 is defined by an XY plane and a vertical direction is defined by a Z-axis direction.

A permanent magnet 110, an accommodating portion 111 that can house the permanent magnet 110, lid portions 113 and 114 that can cover the accommodating portion 111, and a magnet displacing mechanism 115 that has a stage for mounting the accommodating portion 111 and moves the accommodating portion 111 in each of a X-axis direction, a Y-axis direction and the Z-axis direction together with the permanent magnet 110 are provided in the leg portion 102.

The permanent magnet 110 has a rectangular parallelepiped shape, for example, and generates a magnetic field M that acts on the permanent magnet 19 included in the capsule endoscope 10. The permanent magnet 110 is supported inside the accommodating portion 111 so as to be movable along a vertical direction.

The accommodating portion 111 is a box-shaped container of which the upper end is open. The accommodating portion 111 is formed of a ferromagnetic substance such as iron and prevents or suppresses leakage toward a lateral side or a lower side of the drawing, of the magnetic field M generated by the permanent magnet 110.

The lid portions 113 and 114 are provided in the upper-end opening of the accommodating portion 111 so as to be opened and closed. The lid portions 113 and 114 are shielding members which are formed of a ferromagnetic substance such as iron and are closed when the permanent magnet 110 is accommodated in the accommodating portion 111 to thereby prevent or suppress leakage toward an upper side of the drawing, of the magnetic field M generated by the permanent magnet 110.

FIG. 1A illustrates a state in which the lid portions 113 and 114 are open, the permanent magnet 110 is exposed to the outside of the accommodating portion 111, and the magnetic field M is formed in the mounting area of the subject 2. Hereinafter, this state will be referred to as a non-shielding state and the position of the lid portions 113 and 114 at that time will be referred to as a non-shielding position. When the guidance device 100 is in the non-shielding state, by allowing the magnetic field M to act on the permanent magnet 19 included in the capsule endoscope 10, the capsule endoscope 10 can be guided. On the other hand, FIG. 1B illustrates a state in which the permanent magnet 110 is accommodated in the accommodating portion 111, the lid portions 113 and 114 are closed, and the magnetic field M is confined in the accommodating portion 111. Hereinafter, this state will be referred to as a shielding state and the position of the lid portions 113 and 114 at that time will be referred to as a shielding position. When the guidance device 100 is in the shielding state, it is possible to prevent or minimize the action of the magnetic field M on the outside of the accommodating portion 111.

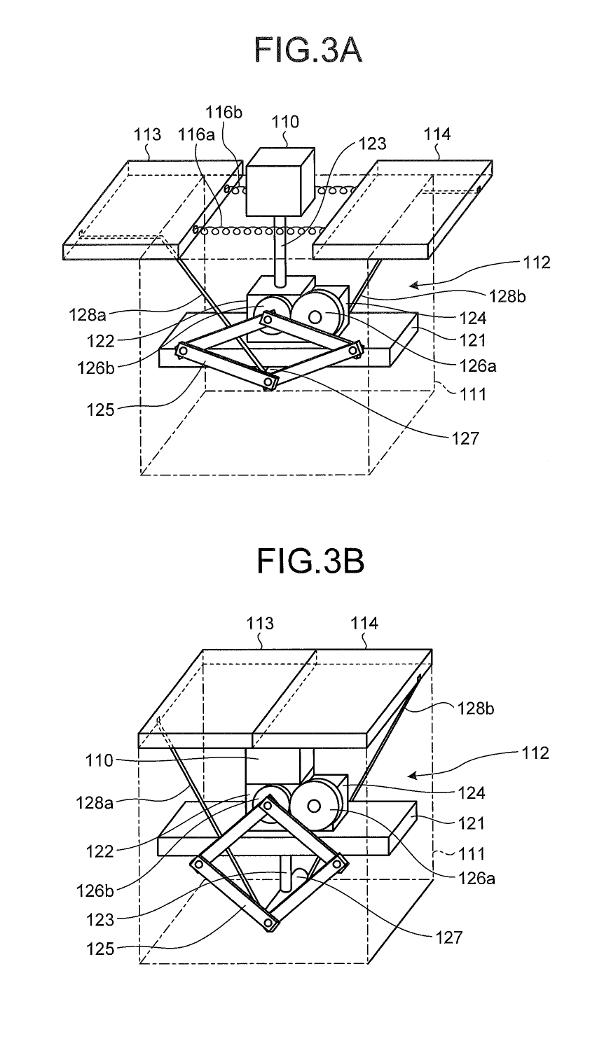

FIGS. 3A and 3B are perspective views schematically illustrating an inner configuration of the permanent magnet 110 and the accommodating portion 111. Among these drawings, FIG. 3A illustrates the non-shielding state and FIG. 3B illustrates the shielding state. A magnet holding mechanism 112 that holds the permanent magnet 110 is provided in the accommodating portion 111. The magnet holding mechanism 112 includes a fixed plate 121 fixed to side walls of the accommodating portion 111, a slide joint 122, a slider 123 that is slidably fitted to the slide joint 122, a motor 124, a link mechanism 125, gears 126a and 126b, a wire holding portion 127, and wires 128a and 128b. Among these constituent elements, the slide joint 122, the slider 123, the link mechanism 125, the gears 126a and 126b, and the wire holding portion 127 form a supporting mechanism that supports the permanent magnet 110 so as to be movable in a vertical direction.

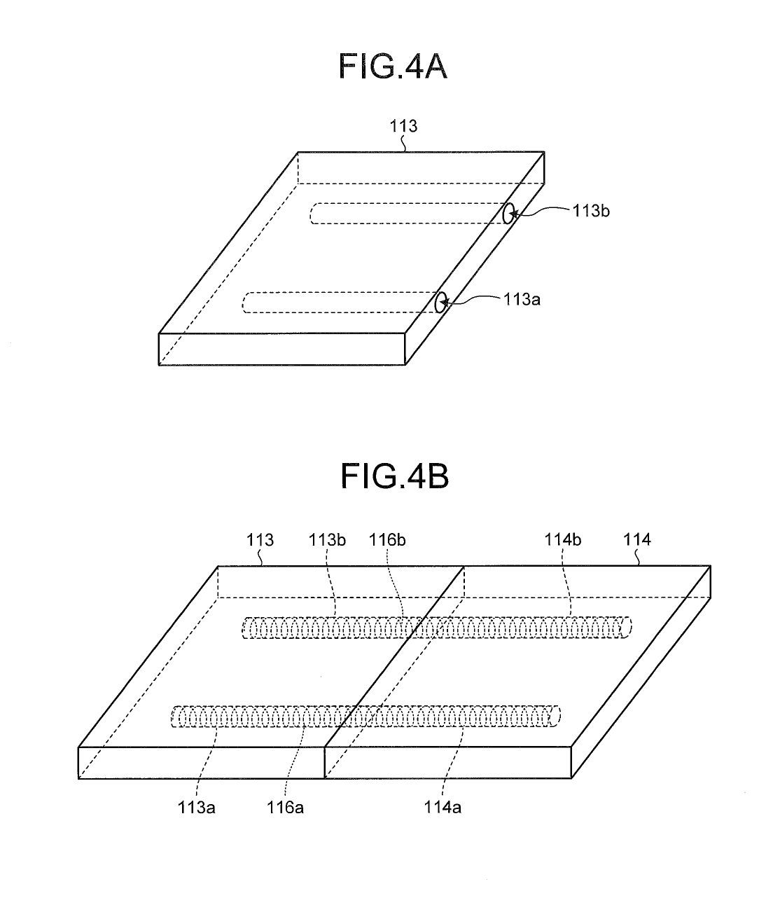

FIGS. 4A and 4B are schematic diagrams for describing the structure of the lid portions 113 and 114. As illustrated in FIG. 4A, bottomed holes 113a and 113b that are open to a surface contacting the lid portion 114 are formed in the lid portion 113. Bottomed holes that are open to a surface contacting the lid portion 113 are also formed in the lid portion 114. As illustrated in FIG. 4B, the holes 113a and 113b formed in the lid portion 113 and the holes 114a and 114b formed in the lid portion 114 are aligned so that the positions of the openings match on the contacting surfaces of the lid portions 113 and 114.

A coil spring 116a is inserted in the hole 113a of the lid portion 113 and the hole 114a of the lid portion 114. Each of ends of the coil spring 116a is fixed to the respective bottoms of the holes 113a and 114a. Similarly, a coil spring 116b is inserted in the hole 113b of the lid portion 113 and the hole 114b of the lid portion 114. Each of ends of the coil spring 116b is fixed to the respective bottoms of the holes 113b and 114b. With these coil springs 116a and 116b, the lid portions 113 and 114 are biased so as to move from the shielding state to the non-shielding state. As illustrated in FIGS. 3A and 3B, the wires 128a and 128b and the coil springs 116a and 116b form a shielding member moving mechanism that moves the lid portions 113 and 114 in conjunction with the movement of the permanent magnet 110 in the vertical direction.

An opening is formed in an approximately central portion of the fixed plate 121 and the slide joint 122 is provided on the opening. The slider 123 is provided so as to be able to pass through the opening.

The permanent magnet 110 is fixed to an upper end of the slider 123. The permanent magnet 110 moves in a vertical direction when the slider 123 slides with respect to the slide joint 122.

The motor 124 is fixed to an upper surface of the fixed plate 121. The motor 124 is a driving unit that operates with the electric power supplied from the power source unit 105 (see FIG. 1A) and moves the permanent magnet 110 in the vertical direction. The gears 126a and 126b transmit the driving force of the motor 124 to one end of the link mechanism 125. The wire holding portion 127 is connected to a lower end of the slider 123 and the other end of the link mechanism 125.

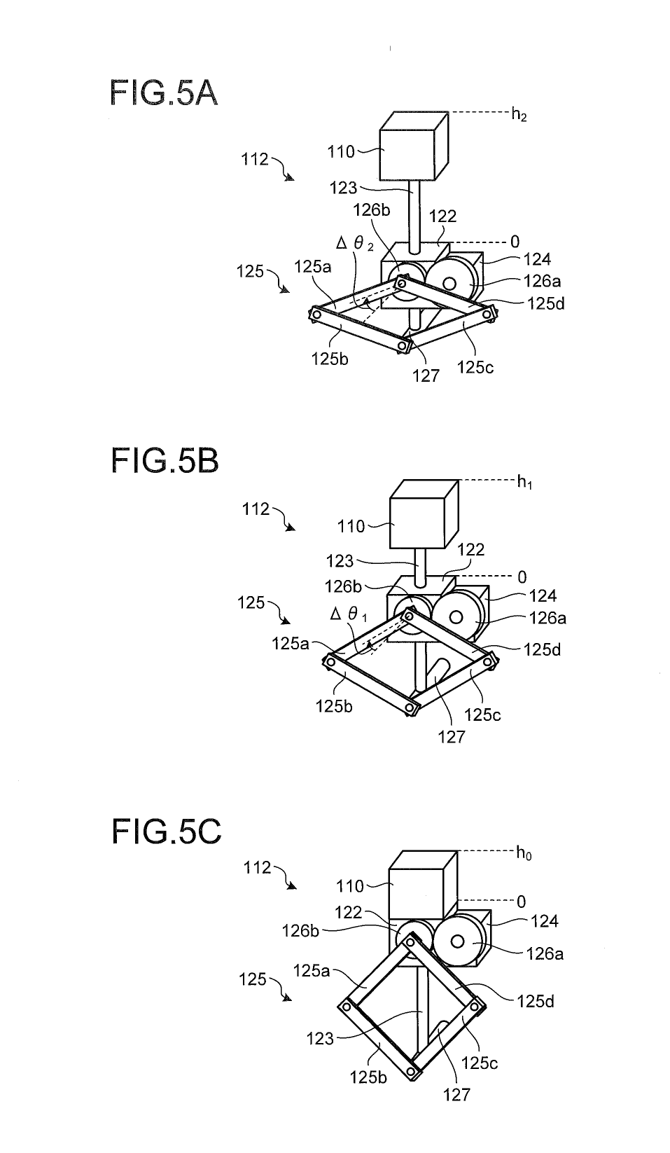

FIGS. 5A to 5C are perspective views for describing the operation of the magnet holding mechanism 112. The link mechanism 125 has a rhombus structure in which four links 125a to 125d are joined. When the motor 124 is driven to rotate the link 125a by a predetermined angle with the aid of the gears 126a and 126b, the other links 125b to 125d move in an interlocked manner and the position of the wire holding portion 127 is moved up and down. As a result, the slider 123 connected to the wire holding portion 127 slides and the permanent magnet 110 moves in a vertical direction.

For example, as illustrated in FIG. 5C, the angle of the link 125a when the permanent magnet 110 is at the lowest position h₀ within a movable range is defined as a reference angle. When the link 125a is rotated by an angle Δθ₀ from this state as illustrated in FIG. 5B, the permanent magnet 110 rises up to a height h₁. Moreover, when the link 125a is rotated further by a larger angle Δθ₂ as illustrated in FIG. 5A, the permanent magnet 110 rises up to a height h₂. The heights h₀ to h₂ are the heights measured from the upper surface of the slide joint 122.

As illustrated in FIGS. 3A and 3B, one set of ends of the wires 128a and 128b are fixed to the outer ends of the lid portions 113 and 114, respectively. Moreover, the other set of ends of the wires 128a and 128b are fixed to the wire holding portion 127. As illustrated in FIG. 3A, when the wire holding portion 127 is positioned on the upper side, the permanent magnet 110 is exposed outside the accommodating portion 111 and the wires 128a and 128b are in a loose state. Thus, the lid portions 113 and 114 are expanded toward the outer side by the biasing force of the coil springs 116a and 116b to enter an open state. This state is the non-sliding state in which the magnetic field M generated by the permanent magnet 110 is formed up to the mounting area of the subject 2 (see FIG. 1A).

On the other hand, as illustrated in FIG. 3B, when the wire holding portion 127 is positioned on the lower side, the permanent magnet 110 is accommodated in the accommodating portion 111 and the lid portions 113 and 114 are pulled by the wires 128a and 128b to enter a closed state. This state is the shielding state in which the magnetic field M generated by the permanent magnet 110 is confined in the accommodating portion 111 (see FIG. 1B).

In general, the guidance of the capsule endoscope 10 is performed in a state in which the permanent magnet 110 is raised to a predetermined height h₂ so as to approach the bed 101 as much as possible. While the guidance of the capsule endoscope 10 is being performed, the motor 124 maintains the permanent magnet 110 at the height h₂ against the gravity acting on the permanent magnet 110 and the coil springs 116a and 116b maintain a state in which the lid portions 113 and 114 of the permanent magnet 110 are being opened. Moreover, when the guidance of the capsule endoscope 10 is not performed, the motor 124 moves the permanent magnet 110 to the lowest position h₀ and closes the lid portions 113 and 114 by allowing the lid portions 113 and 114 to be pulled by the wires 128a and 128b moving in conjunction with this movement of the permanent magnet 110. As a result, the permanent magnet 110 is accommodated in the accommodating portion 111.

Referring again to FIGS. 1A and 1B, the magnet displacing mechanism 115 includes an X-axis stage 131 that is movable along an X-axis direction, an X-axis driving motor 132 that drives the X-axis stage 131, a Y-axis stage 133 that is movable along a Y-axis direction, a Y-axis driving motor 134 that drives the Y-axis stage 133, a Z-axis stage 135 that is movable in a Z-axis direction, and a Z-axis driving motor 136 that drives the Z-axis stage 135. When the permanent magnet 110 is moved by such a magnet displacing mechanism 115 together with the accommodating portion 111, the magnetic field M acting on the permanent magnet 19 in the capsule endoscope 10 changes. As a result, the capsule endoscope 10 introduced into the subject 2 can be guided.

The Z-axis stage 135 is attached to a ball screw 137 provided between the bottom surface of the leg portion 102 and the lower surface of the bed 101. When the Z-axis driving motor 136 is driven to rotate the ball screw 137, the Z-axis stage 135 moves along the Z-axis direction. The movable range of the Z-axis stage 135 is set such that the capsule endoscope 10 can be guided when the permanent magnet 110 is maintained at the height h₂.

The X-axis stage 131 is attached to the Z-axis stage 135 via a slide rail 138 in which a rack-and-pinion mechanism is included. The X-axis stage 131 moves along the X-axis direction with respect to the Z-axis stage 135 when the X-axis driving motor 132 is driven.

The Y-axis stage 133 is attached to the X-axis stage 131 via a slide rail (not illustrated) in which a rack-and-pinion mechanism is included. The Y-axis stage 133 moves along the Y-axis direction in relation to the X-axis stage 131 when the Y-axis driving motor 134 is driven.

The operation input unit 103 is configured as an input device like a joystick, for example, that can be operated for tilt motion in an up-down direction and a left-right direction and inputs a signal corresponding to an operation of a user's operation performed on the operation input unit 103 to the control unit 104. Specifically, an instruction signal that indicates the start and the end of magnetic guidance of the capsule endoscope 10, an instruction signal that indicates the position and the direction for magnetically guiding the capsule endoscope 10, and other signals are input from the operation input unit 103 to the control unit 104. The configuration of the operation input unit 103 is not limited to the joystick, but may be an input device such as various buttons or an operating lever and a pointing device such as a mouse or a touch panel.

The control unit 104 includes a shielding state controller 141 that controls the operation of the motor 124 of the magnet holding mechanism 112, an X-axis drive controller 142 that controls the operation of the X-axis driving motor 132, a Y-axis drive controller 143 that controls the operation of the Y-axis driving motor 134, and a Z-axis drive controller 144 that controls the operation of the Z-axis driving motor 136. The control unit 104 performs control of allowing the guidance device 100 to transition between the shielding state and the non-shielding state and control of moving the X-axis stage 131, the Y-axis stage 133, and the Z-axis stage 135 so that the capsule endoscope 10 in the subject 2 can be guided to a position and a direction desired by the user according to the signal input by the operation input unit 103.

The power source unit 105 supplies electric power for operating the control unit 104, the motor 124 of the magnet holding mechanism 112, and the X-axis driving motor 132, the Y-axis driving motor 134, and the Z-axis driving motor 136 of the magnet displacing mechanism 115 to these respective units.

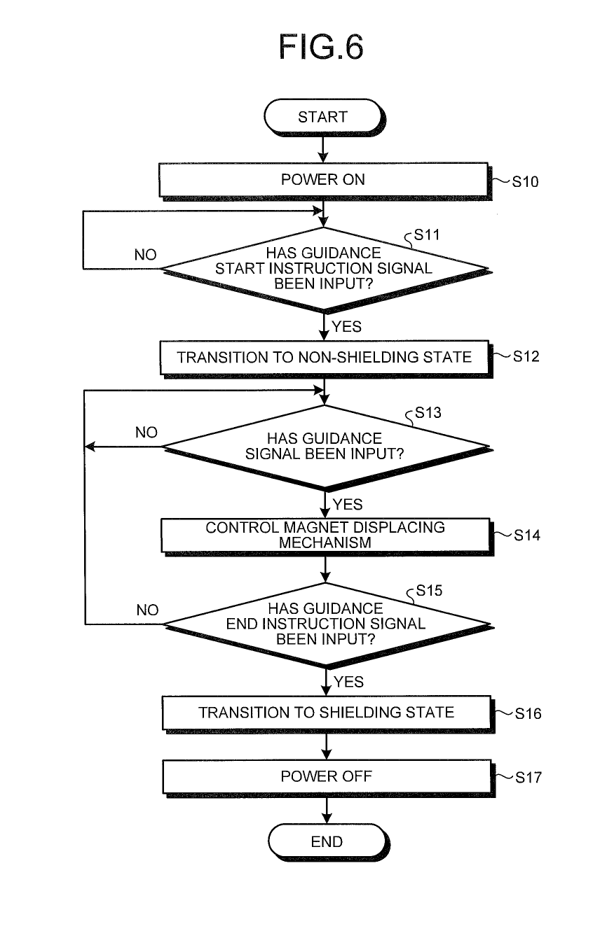

Next, the operation of the guidance device 100 will be described. FIG. 6 is a flowchart illustrating the operation of the guidance device 100. In a period in which the power of the guidance device 100 is turned off, in the guidance device 100, the permanent magnet 110 is positioned at the lowest position h₀ and the lid portions 113 and 114 are closed in the shielding state (see FIG. 3B and FIG. 5C).

When the power of the guidance device 100 is turned on in step S10, the control unit 104 determines whether an instruction signal that indicates the start of guidance of the capsule endoscope 10 is input from the operation input unit 103 (step S11). When the instruction signal is not input (step S11: No), the control unit 104 waits until the instruction signal is input.

On the other hand, when the instruction signal for instructing the start of guidance of the capsule endoscope 10 is input (step S11: Yes), the shielding state controller 141 perform control to put the guidance device 100 into the non-shielding state (step S12). That is, the motor 124 is rotated by a predetermined amount so that the permanent magnet 110 is moved upward to the height h₂ at which the capsule endoscope 10 can be guided. Since the wires 128a and 128b loosen in conjunction with the upward movement, the lid portions 113 and 114 are opened by the biasing force of the coil springs 116a and 116b (see FIG. 3A and FIG. 5A).

Subsequently, in step S13, the control unit 104 determines whether an instruction signal (guidance signal) for guiding the capsule endoscope 10 is input from the operation input unit 103. When the guidance signal is not input (step S13: No), the control unit 104 waits until the guidance signal is input.

On the other hand, when the guidance signal for the capsule endoscope 10 is input (step S13: Yes), the control unit 104 controls the magnet displacing mechanism 115 to change the position of the permanent magnet 110 (step S14). Specifically, the control unit 104 calculate a movement amount and a moving direction of the permanent magnet 110 necessary for moving the capsule endoscope 10 to a position and a direction desired by the user based on the guidance signal input by the operation input unit 103 and operates the X-axis driving motor 132, the Y-axis driving motor 134, and the Z-axis driving motor 136 according to the calculation result. As a result, the permanent magnet 110 is moved in the XYZ directions with the aid of the accommodating portion 111 and the magnet holding mechanism 112. As a result, the magnetic field M acting on the permanent magnet 19 included in the capsule endoscope 10 changes and the position and the direction of the capsule endoscope 10 in the subject 2 can be changed.

Subsequently, in step S15, the control unit 104 determines whether an instruction signal that indicates the end of the guidance of the capsule endoscope 10 is input from the operation input unit 103. When the instruction signal is not input (step S15: No), the operation of the guidance device 100 returns to step S13.

When the instruction signal that indicates the end of guidance of the capsule endoscope 10 is input (step S15: Yes), the shielding state controller 141 performs control to put the guidance device 100 into the shielding state (step S16). That is, the motor 124 is rotated by a predetermined amount so that the permanent magnet 110 is moved downward to the lowest position h₀. The lid portions 113 and 114 are closed by being pulled by the wires 128a and 128b that is interlocked with the downward movement (see FIG. 3B and FIG. 5C). Moreover, simultaneously with this, control is performed to operate the X-axis driving motor 132, the Y-axis driving motor 134, and the Z-axis driving motor 136 so that the X-axis stage 131, the Y-axis stage 133, and the Z-axis stage 135 return to the same initial positions at those during the power-on of the guidance device 100.

After that, in step S17, when the power of the guidance device 100 is turned off, the operation of the guidance device 100 ends.

Subsequently, the operation of the capsule medical device guidance system 1 when an unsuspected situation such as an unexpected power failure or an earthquake occurs will be described. In such a case, it is necessary to immediately shield the magnetic force generated by the permanent magnet 110 in order to prevent or minimize the leakage of the magnetic field from the guidance device 100.

When an unexpected power failure occurs in a state in which the guidance device 100 is in the non-shielding state, the electric power supply from the power source unit 105 to the guidance device 100 stops. In this way, the driving operation of the motor 124 of the magnet holding mechanism 112 stops and the gears 126a and 126b enter a freely rotating state. As a result, the permanent magnet 110 falls up to the lowest position h₀ due to its own weight, the lid portions 113 and 114 are closed in conjunction with the falling of the permanent magnet, and the guidance device 100 enters the shielding state.

Moreover, when an earthquake or the like occurs in a state the guidance device 100 is in the non-shielding state, the user may turn off the power of the guidance device 100. In this way, the permanent magnet 110 falls, the lid portions 113 and 114 are closed, and the guidance device 100 enters the shielding state similarly to the case of the power failure. Alternatively, a vibration detection unit may be provided in the guidance device 100 so that the power of the guidance device 100 is automatically turned off when a vibration of a predetermined magnitude or larger occurs.

As described above, in the first embodiment, the wires 128a and 128b that open and close the lid portions 113 and 114 in conjunction with the movement of the permanent magnet 110 in the vertical direction are provided so that during execution of the guidance of the capsule endoscope 10, the permanent magnet 110 is maintained at the height h₂ at which the capsule endoscope 10 can be guided against the gravity acting on the permanent magnet 110 by the driving force of the motor 124 and the lid portions 113 and 114 are maintained at the non-shielding position. Due to this, when the electric power supply to the motor 124 stops in this state, the motor 124 loses the driving force, the permanent magnet 110 falls with its own weight, and the lid portions 113 and 114 are moved to the shielding position. Thus, even when an unsuspected situation such as an unexpected power failure occurs, it is possible to reliably shield the magnetic field M generated by the permanent magnet 110 and to secure the safety of the guidance device 100.

In the first embodiment described above, a damper may be provided in the slide joint 122 so that the permanent magnet 110 can fall smoothly when the gears 126a and 126b are in the freely rotating state.

Moreover, in the first embodiment described above, a posture changing mechanism for changing the posture of the permanent magnet 110 may be further provided in the magnet displacing mechanism 115. In this way, it is possible to control the position and the posture of the permanent magnet 110. As a result, control of changing the direction of the capsule endoscope 10 can be performed more precisely.

### Modified Example 1-1

Next, Modified Example 1-1 of the first embodiment of the present invention will be described.

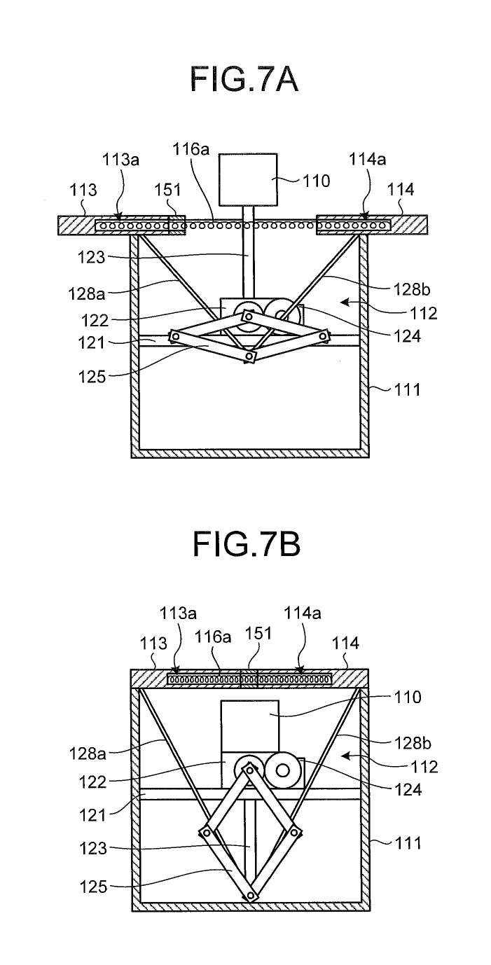

FIGS. 7A and 7B are schematic diagrams illustrating a configuration of an accommodating portion provided in a guidance device according to Modified Example 1-1. Among these drawings, FIG. 7A illustrates the state of the accommodating portion when the guidance device is in the non-shielding state and FIG. 7B illustrates the state of the accommodating portion when the guidance device is in the shielding state. As illustrated in FIGS. 7A and 7B, in Modified Example 1-1, an impact absorbing member 151 formed of an elastic member such as rubber, for example, is further provided on an end surface of the lid portion 113 of the accommodating portion 111. In this case, the holes 113a and 113b in which the coil springs 116a and 116b are accommodated are formed so as to pass through the impact absorbing member 151.

Here, in the first embodiment described above, when the shielding state is created when the electric power supply (the driving of the motor 124) stops, the lid portions 113 and 114 may accelerate due to the magnetic attracting force of the permanent magnet 110. Thus, when the impact absorbing member 151 is provided on one or both contacting surfaces (end surfaces) of the lid portions 113 and 114, it is possible to absorb the impact when both make contact with each other even when the lid portions 113 and 114 accelerate. In this way, it is possible to prevent damage of the lid portions 113 and 114 and the accommodating portion 111 and to suppress the occurrence of vibration and noise to thereby perform examination safely. Moreover, when the impact absorbing member 151 is configured as a replaceable component, it is possible to extend the service life of the guidance device.

### Modified Example 1-2

Next, Modified Example 1-2 of the first embodiment of the present invention will be described.

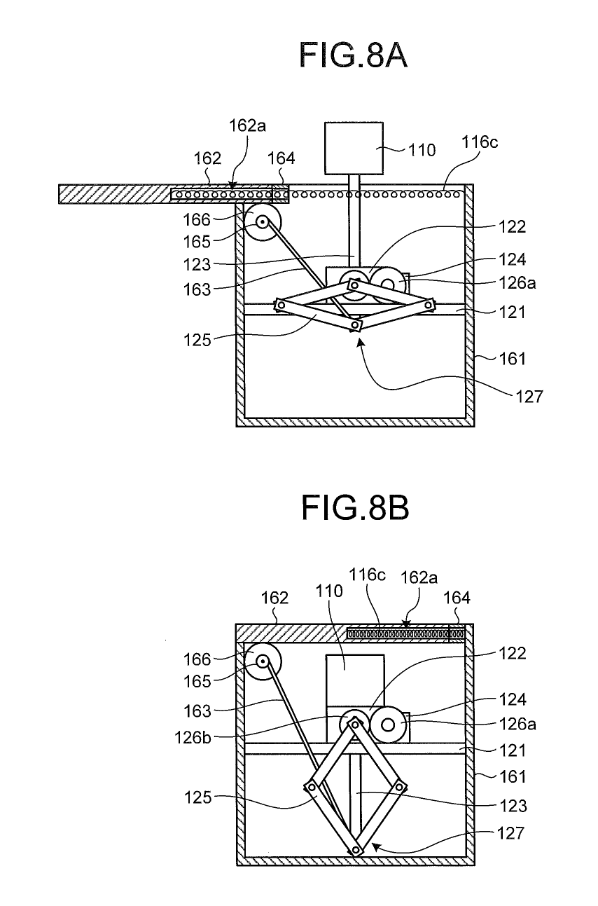

FIGS. 8A and 8B are schematic diagrams illustrating a configuration of an accommodating portion provided in a guidance device according to Modified Example 1-2. As illustrated in FIGS. 8A and 8B, the guidance device according to Modified Example 1-2 includes an accommodating portion 161 and a lid portion 162 having a single-door structure instead of the accommodating portion 111 and the lid portions 113 and 114 having a double-door structure illustrated in FIGS. 1A and 1B. A wire 163 having one end fixed to the wire holding portion 127 is connected to the lid portion 162. The structure of the supporting mechanism (the slide joint 122 to the wire holding portion 127) that supports the permanent magnet 110 so as to be movable in the vertical direction is the same as that of the first embodiment. Moreover, an impact absorbing member 164 that absorbs the impact when the lid portion 162 makes contact with the accommodating portion 161 is provided at the end of the lid portion 162. Further, a bottomed hole 162a that is open to a surface contacting an inner wall of the accommodating portion 161 is formed in the lid portion 162 and the impact absorbing member 164. A coil spring 116c is disposed in the hole 162a. One end of the coil spring 116c is fixed to the bottom of the hole 162a and the other end is fixed to the inner wall of the accommodating portion 161.

A pulley 165 around which the wire 163 is wound and a roller 166 attached to the pulley 165 so as to be rotatable together with the pulley 165 are provided near the upper end of the accommodating portion 161. A portion of the outer circumferential surface of the roller 166 is in contact with the lower surface of the lid portion 162 so that the lid portion 162 slides when the roller 166 rotates.

As illustrated in FIG. 8A, when the wire holding portion 127 moves upward, since the wire 163 enters a loose state, the lid portion 162 is slid to be open by the biasing force of the coil spring 116c. With sliding of the lid portion 162, the roller 166 and the pulley 165 rotate to roll up the wire 163. On the other hand, as illustrated in FIG. 8B, when the wire holding portion 127 moves downward, the pulley 165 and the roller 166 rotate by being pulled by the wire 163, and the lid portion 162 is slid to be open with rotation of the roller 166.

According to Modified Example 1-2, since the number of operating units (the lid portion 162) necessary for realizing the transition between the shielding state and the non-shielding state is small, it is possible to simplify the configuration. In Modified Example 1-2, although the impact absorbing member 164 is provided in the lid portion 162, the impact absorbing member 164 may be provided in the accommodating portion 161.

### Modified Example 1-3

Next, Modified Example 1-3 of the first embodiment of the present invention will be described.

In the first embodiment described above, when the electric power supply to the motor 124 stops, the lid portions 113 and 114 are closed in conjunction with the falling of the permanent magnet 110 due to its own weight. However, in this case, as described above, the lid portions 113 and 114 may accelerate due to the magnetic attracting force of the permanent magnet 110 and an impact may be applied due to falling of the permanent magnet 110.

Therefore, in Modified Example 1-3, when the electric power supply to the motor 124 stops, the permanent magnet 110 is moved downward at a controlled speed rather than allowing the permanent magnet 110 to fall. Specifically, an energy storage unit such as a battery may be provided in the motor 124 in addition to the power source unit 105 so that the motor 124 is driven by the energy stored in the energy storage unit to move the permanent magnet 110.

For example, when a battery is provided as the energy storage unit, a circuit is designed so that electric power starts being supplied from the battery to the motor 124 when the electric-power supply from the power source unit 105 to the motor 124 stops abruptly. Moreover, an electrical energy at least necessary for moving the permanent magnet 110 from the height h₂ to the height h₀ (see FIGS. 5A to 5C) may be stored in the battery.

In this way, when the electric power supply from the power source unit 105 to the motor 124 stops abruptly, electric power is supplied from the battery to the motor 124 and the permanent magnet 110 is moved at a controlled speed from the height h₂ to the height h₀ by the driving force of the motor 124. In line with this movement, the lid portions 113 and 114 also move to the shielding position at a speed corresponding to the moving speed of the permanent magnet 110.

### Second Embodiment

Next, the second embodiment of the present invention will be described.

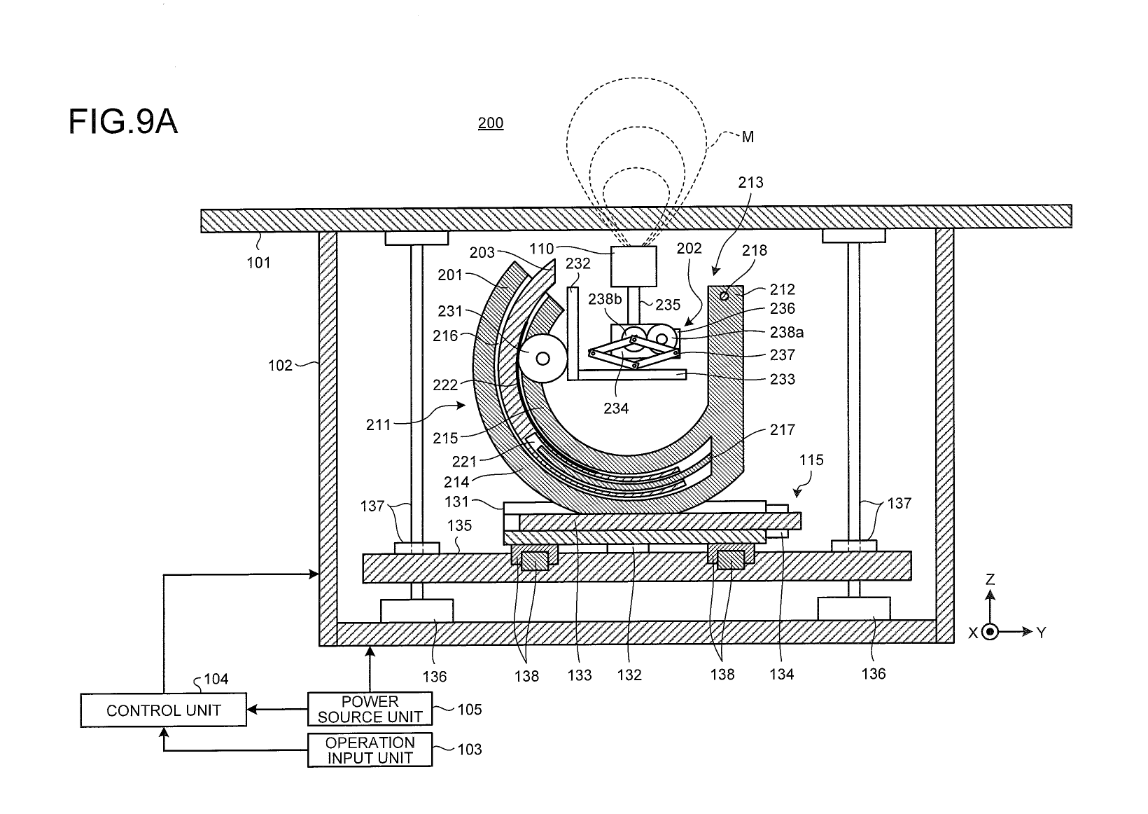

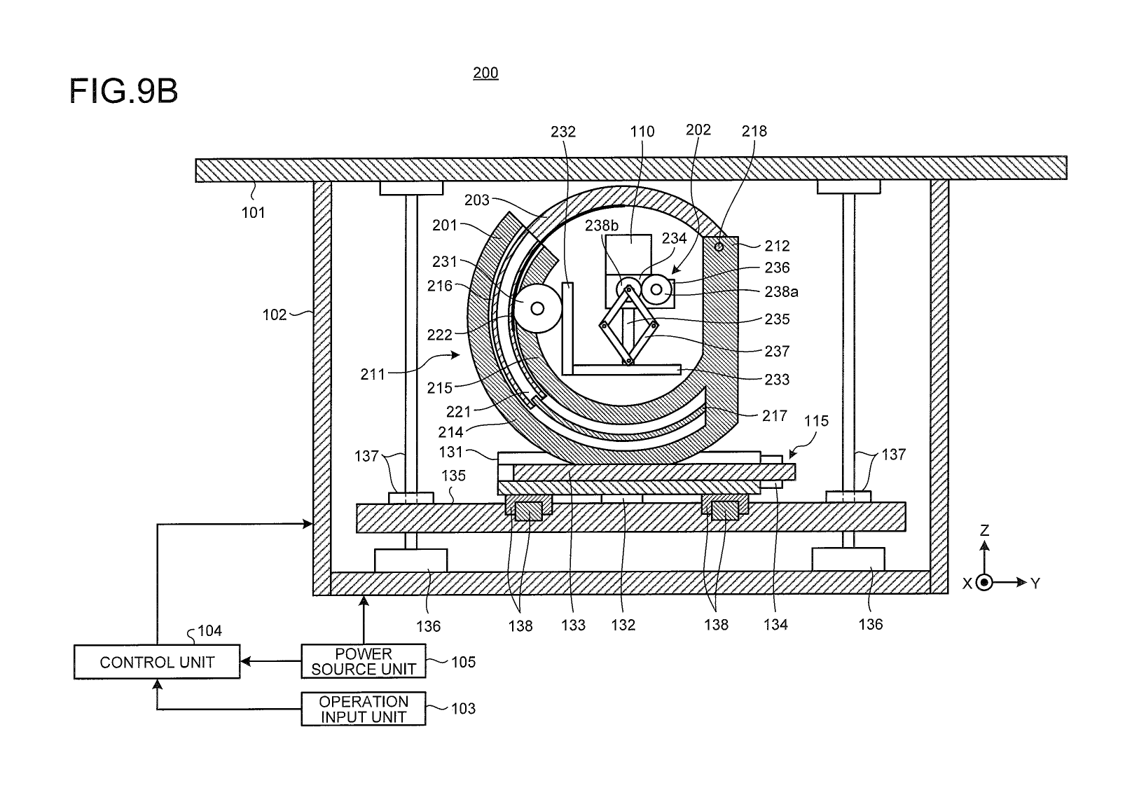

FIGS. 9A and 9B are schematic diagrams illustrating a configuration of a guidance device according to the second embodiment of the present invention. As illustrated in FIG. 9A, a guidance device 200 according to the second embodiment includes an accommodating portion 201, a magnet holding mechanism 202, and a lid portion 203 instead of the accommodating portion 111, the magnet holding mechanism 112, and the lid portions 113 and 114 illustrated in FIGS. 1A and 1B. The respective constituent elements of the guidance device 200 other than the accommodating portion 201, the magnet holding mechanism 202, and the lid portion 203 have the same configuration as those of the first embodiment.

The accommodating portion 201 has generally a cylindrical shape of which both ends (the end surfaces parallel to the drawing surface) are closed, and FIGS. 9A and 9B illustrate a section (a cross-section) orthogonal to the central axis of the accommodating portion 201. Such an accommodating portion 201 is formed of a ferromagnetic substance such as iron and is configured to prevent or minimize the leakage toward the lateral side and the lower side of the drawing, of the magnetic field M generated by the permanent magnet 110.

The accommodating portion 201 has a curved portion 211 of which the cross-section has a circular arc shape and a planar portion 212 connected to one end of the curved portion 211. Moreover, an opening 213 is formed between the other end of the curved portion 211 and the planar portion 212. The curved portion 211 has a double-wall structure and a lid supporting portion 217 of which the cross-section is curved to form a circular arc that is concentric to the curved portion 211 is provided in a gap 216 between an outer wall 214 and an inner wall 215 of the curved portion 211. One end of the lid supporting portion 217 is fixed to the planar portion 212.

The lid portion 203 has a cross-section having a circular arc shape and has a curved shape that is protruded to the outer side in relation to the accommodating portion 201 and is provided so as to be opened and closed in relation to the opening 213 of the accommodating portion 201. The lid portion 203 is a shielding member which is formed of a ferromagnetic substance such as iron and which prevents or minimizes the leakage toward the upper side of the drawing, of the magnetic field M generated by the permanent magnet 110 by being closed when the permanent magnet 110 is accommodated in the accommodating portion 201.

A slit 221 that follows the curve of the lid portion 203 is formed inside the lid portion 203 so that the lid portion 203 is supported by the accommodating portion 201 when the lid supporting portion 217 is inserted into the slit 221. By supporting the lid portion 203 in this manner, the lid portion 203 can move between the shielding position and the non-shielding position while drawing a circular arc-shaped trajectory having the same curvature as the cross-section thereof. Further, a driving belt 222 is provided on an inner circumferential surface of the lid portion 203.

FIG. 9A illustrates the non-shielding state in which the lid portion 203 is open. In the non-shielding state, the lid supporting portion 217 is inserted deep into the slit 221, and the lid portion 203 is accommodated at a deep position (the non-shielding position) of the gap 216 of the curved portion 211. On the other hand, FIG. 9B illustrates the shielding state in which the lid portion 203 is closed. In the shielding state, the lid supporting portion 217 is inserted shallow into the slit 221, and the lid portion 203 is accommodated at a shallow position (the shielding position) of the gap 216.

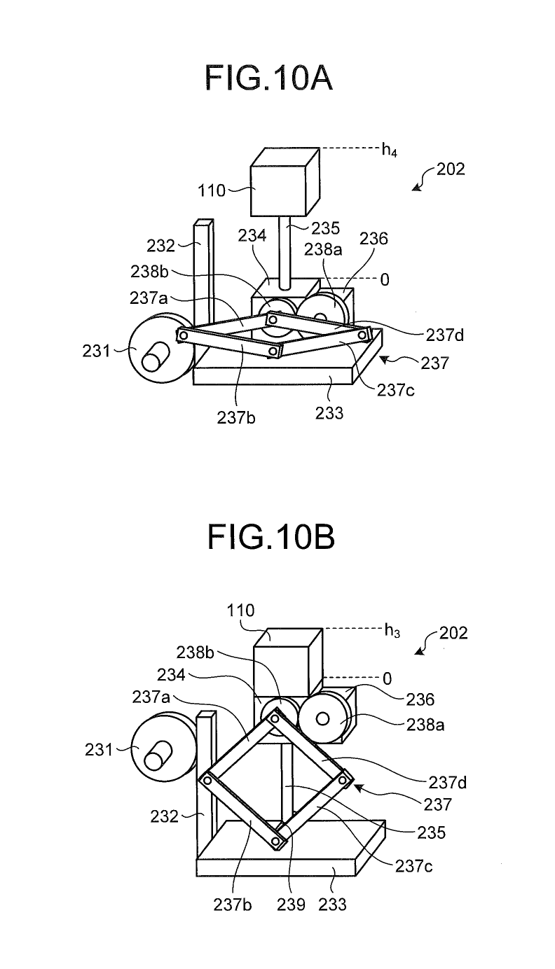

FIGS. 10A and 10B are perspective views illustrating the magnet holding mechanism 202 at an enlarged scale. The magnet holding mechanism 202 includes a pinion 231 that engages with the driving belt 222 provided in the lid portion 203, a rack 232 that engages with the pinion 231, a fixed plate 233 fixed to the rack 232, a slide joint 234, a slider 235, a motor 236 attached to the slide joint 234, a link mechanism 237, gears 238a and 238b that transmit power of the motor 236 to the link mechanism 237, and a fixed portion 239 fixed to the fixed plate 233. Among these constituent elements, the slide joint 234, the slider 235, the link mechanism 237, the gears 238a and 238b, and the fixed portion 239 form a supporting mechanism that supports the permanent magnet 110 so as to be movable in a vertical direction. Moreover, the pinion 231, the rack 232, and the fixed portion 239 form a shielding member moving mechanism that moves the lid portion 203 in conjunction with the movement of the permanent magnet 110 in the vertical direction.

The pinion 231 is fixed at a predetermined position of the inner wall 215 of the accommodating portion 201 and rotates at the position to open and close the lid portion 203 with the aid of the driving belt 222. The rack 232 is provided so as to be movable in a vertical direction. The fixed plate 233 is fixed to the rack 232 and moves in a vertical direction together with the rack 232.

The slide joint 234 and the motor 236 are fixed at a predetermined position in the accommodating portion 201 by a supporting unit (not illustrated).

The permanent magnet 110 is fixed to the upper end of the slider 235. On the other hand, the other end of the slider 235 is fixed to the fixed plate 233 with the fixed portion 239 interposed. When the slider 235 slides in relation to the slide joint 234, the permanent magnet 110, the fixed plate 233, and the rack 232 move in a vertical direction.

The link mechanism 237 has a rhombus structure in which four links 237a to 237d are joined. One end of the link mechanism 237 is connected to the gear 238a. Moreover, the other end of the link mechanism 237 is connected to the fixed portion 239. When the motor 236 is driven to rotate the link 237a by a predetermined angle with the aid of the gears 238a and 238b, the other links 237b to 237d move in an interlocked manner and the fixed plate 233 and the rack 232 move in a vertical direction. As a result, the pinion 231 rotates to move the lid portion 203 along a circular arc. Moreover, the slider 235 also moves in the vertical direction together with the fixed plate 233. As a result, the permanent magnet 110 attached to the upper end of the slider 235 also moves in the vertical direction.

In general, the guidance of the capsule endoscope 10 is performed in a state in which the permanent magnet 110 is raised to a predetermined height h₄ so as to approach the bed 101 as much as possible. While the guidance of the capsule endoscope 10 is being performed, the motor 236 is driven against the gravity acting on the permanent magnet 110 to maintain the permanent magnet 110 at the height h₄ and maintains the lid portion 203 in the open state. Moreover, when the guidance of the capsule endoscope 10 is not performed, the motor 236 moves the permanent magnet 110 to the lowest position h₃ and closes the lid portion 203 in conjunction with this movement. As a result, the permanent magnet 110 is accommodated in the accommodating portion 201. The heights h₃ and h₄ are the heights measured from the upper surface of the slide joint 234.

A lock pin 218 for locking the end of the lid portion 203 may be provided on the upper side of the planar portion 212. The lock pin 218 locks the lid portion 203 when the lid portion 203 is at the shielding position and unlocks the lid portion 203 so that the lid portion 203 can be opened and closed when the motor 236 starts driving and the permanent magnet 110 starts rising. Moreover, the lock pin 218 locks the lid portion 203 again when the permanent magnet 110 falls up to the lowest position h₃. However, the lock pin 218 may be configured so that the locked state can be manually released.

Next, the operation of the guidance device 200 will be described. The operation of the guidance device 200 is generally the same as that of the first embodiment (see FIG. 6) and the operation (step S12) of transitioning from the shielding state to the non-shielding state and the operation (step S16) of transitioning from the non-shielding state to the shielding state are different from those of the first embodiment.

That is, when the transition from the shielding state to the non-shielding state is to be realized (step S12), the motor 236 is rotated by a predetermined amount so that the permanent magnet 110 is moved upward to the height h₄ at which the guidance of the permanent magnet 19 included in the capsule endoscope 10 can be realized. The rack 232 rises in conjunction with the upward movement and the rack 232 rotates the pinion 231. The driving belt 222 is moved with the rotation of the pinion 231 to open the lid portion 203 (see FIG. 9A and FIG. 10A).

On the other hand, when the transition from the non-shielding state to the shielding state is to be realized (step S16), the motor 236 is rotated by a predetermined amount so that the permanent magnet 110 moves downward to the lowest position h₃. The rack 232 falls in an interlocked manner with the downward movement and the rack 232 rotates the pinion 231. The driving belt 222 is moved with the rotation of the pinion 231 to close the lid portion 203 (see FIG. 9B and FIG. 10B).

When an unexpected power failure occurs in a state in which the guidance device 200 is in the non-shielding state, the electric power supply to the respective units of the guidance device 200 stops. In this way, the driving operation of the motor 236 stops and the gears 238a and 238b enter a freely rotating state. As a result, the permanent magnet 110 falls up to the lowest position h₃ due to its own weight, the lid portion 203 is closed in conjunction with the falling of the permanent magnet, and the guidance device 200 enters the shielding state.

Moreover, when an earthquake or the like occurs when the guidance device 200 is in the non-shielding state, the user may turn off the power of the guidance device 200. In this way, the permanent magnet 110 falls, the lid portion 203 is closed, and the guidance device 200 enters the shielding state similarly to the case of the power failure. Alternatively, a vibration detection unit may be provided in the guidance device 200 so that the power of the guidance device 200 is automatically turned off when a vibration of a predetermined magnitude or larger occurs.

As described above, according to the second embodiment, during execution of the guidance of the capsule endoscope 10, the permanent magnet 110 is maintained at the height h₄ at which the capsule endoscope 10 can be guided against the gravity acting on the permanent magnet 110 by the driving force of the motor 236 and the lid portion 203 is maintained at the non-shielding position. Due to this, when the electric power supply to the motor 236 stops in this state, the motor 236 loses the driving force, the permanent magnet 110 falls, and at the same time, the lid portion 203 is moved to the shielding position. Thus, even when an unsuspected situation such as an unexpected power failure occurs, it is possible to reliably shield the magnetic field M generated by the permanent magnet 110 and to secure the safety of the guidance device 200.

In the second embodiment, since the lid portion 203 moves while drawing a circular arc, it is possible to always maintain a certain distance or longer between the lid portion 203 and the permanent magnet 110. Thus, it is possible to prevent the lid portion 203 from accelerating due to the magnetic attracting force of the permanent magnet 110 during the movement of the lid portion 203 and to secure the safety of an operator during assembling or maintenance of the guidance device 200.

### Modified Example 2-1

Next, Modified Example 2-1 of the second embodiment of the present invention will be described.

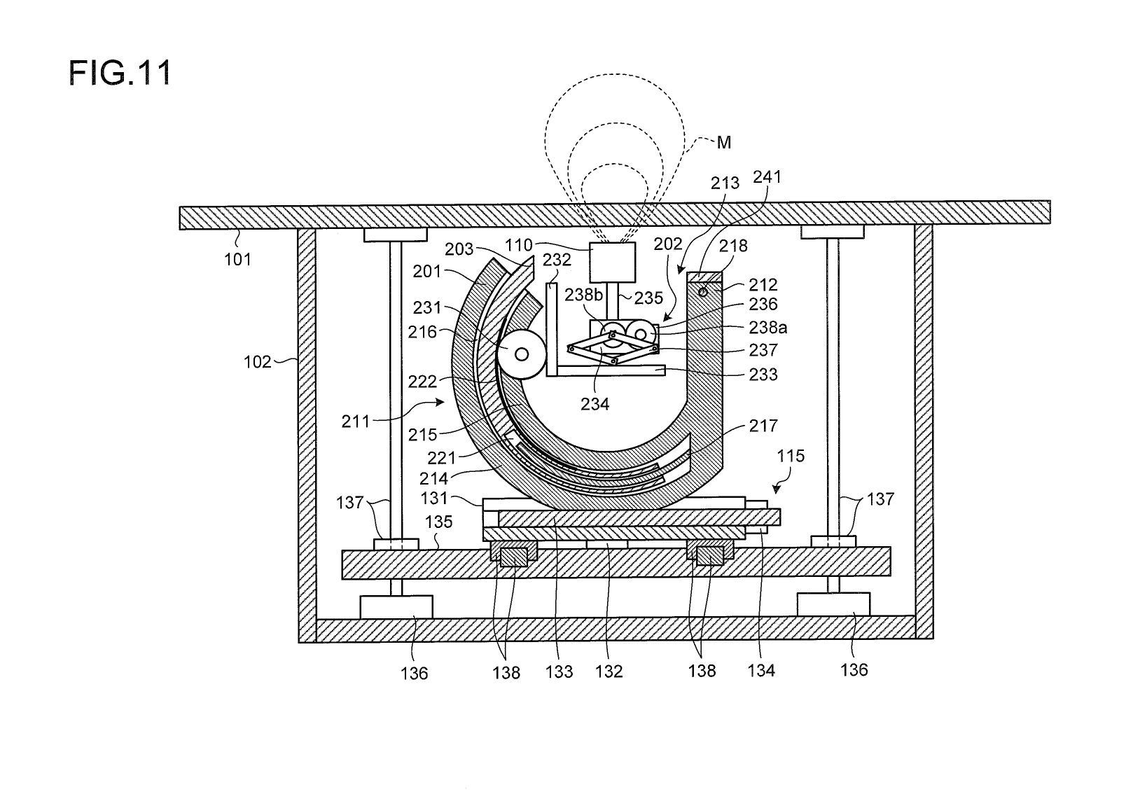

FIG. 11 is a schematic diagram illustrating a configuration of a guidance device according to Modified Example 2-1. As illustrated in FIG. 11, the guidance device according to Modified Example 2-1 has a configuration in which an impact absorbing member 241 formed of an elastic member such as rubber, for example, is further provided at a position at which the lid portion 203 makes contact with the planar portion 212 in the guidance device 200 illustrated in FIGS. 9A and 9B. By providing such an impact absorbing member 241, it is possible to absorb the impact when the lid portion 203 makes contact with the planar portion 212 to prevent damage of the lid portion 203 and the planar portion 212 to suppress the occurrence of vibration and noise to thereby perform examination safely. Moreover, when the impact absorbing member 241 is configured as a replaceable component, it is possible to extend the service life of the guidance device 200. The impact absorbing member may be provided in the lid portion 203 rather than the planar portion 212 and may be provided in both portions.

### Modified Example 2-2

Next, Modified Example 2-2 of the second embodiment of the present invention will be described.

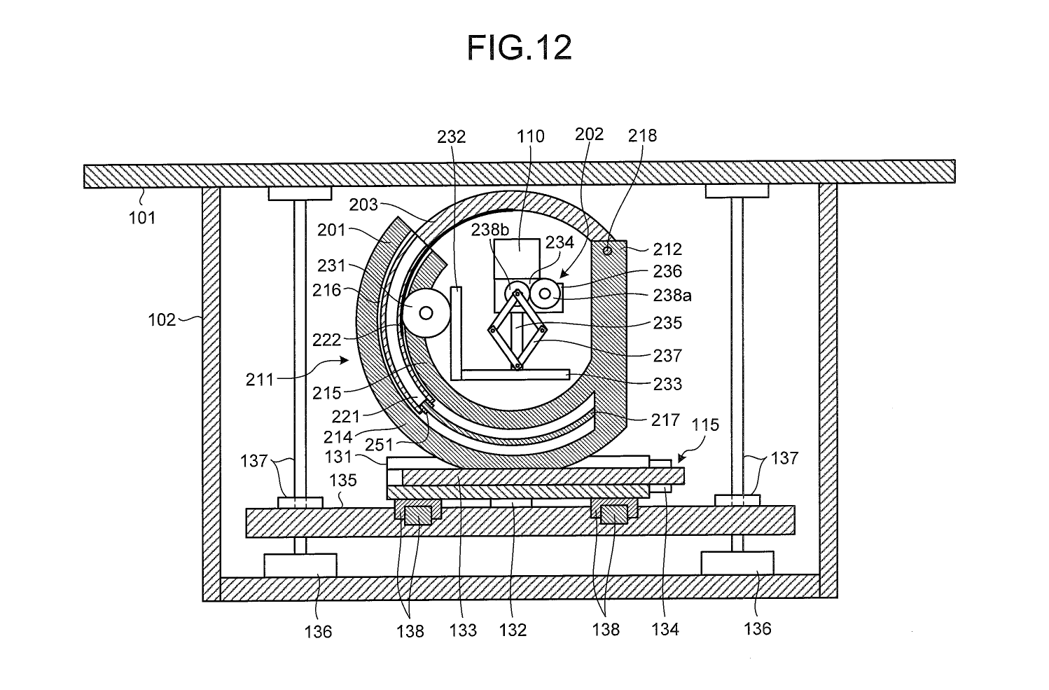

FIG. 12 is a schematic diagram illustrating a configuration of a guidance device according to Modified Example 2-2. As illustrated in FIG. 12, the guidance device according to Modified Example 2-2 has a configuration in which a damper 251 is further provided in the guidance device 200 illustrated in FIGS. 9A and 9B. The damper 251 is a rubber-like member attached to the distal end of the lid supporting portion 217 and has substantially the same outer diameter as an inner diameter of the slit 221.

When such a damper 251 is provided, the movement of the lid portion 203 is stopped by the friction between the inner circumferential surface of the slit 221 and the outer circumferential surface of the damper 251 during the movement of the lid portion 203. Due to this, it is possible to prevent the lid portion 203 from accelerating due to the magnetic attracting force of the permanent magnet 110 during the movement of the lid portion 203 and to secure the safety of an operator during assembling or maintenance of the guidance device 200.

### Modified Example 2-3

Next, Modified Example 2-3 of the second embodiment of the present invention will be described.

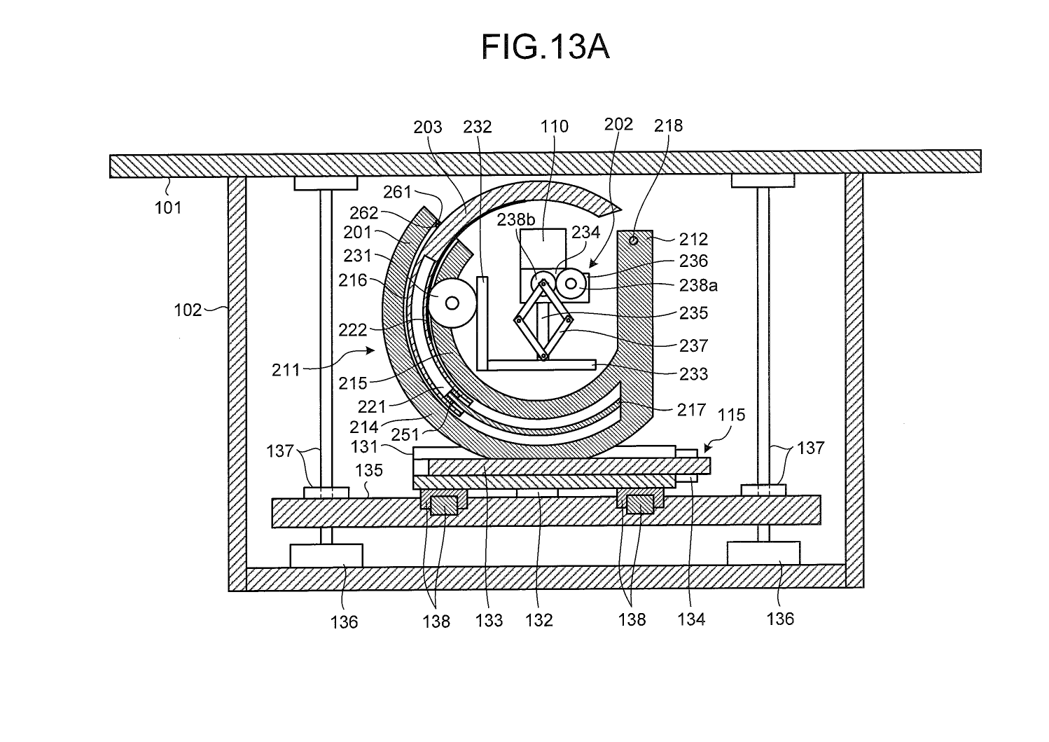

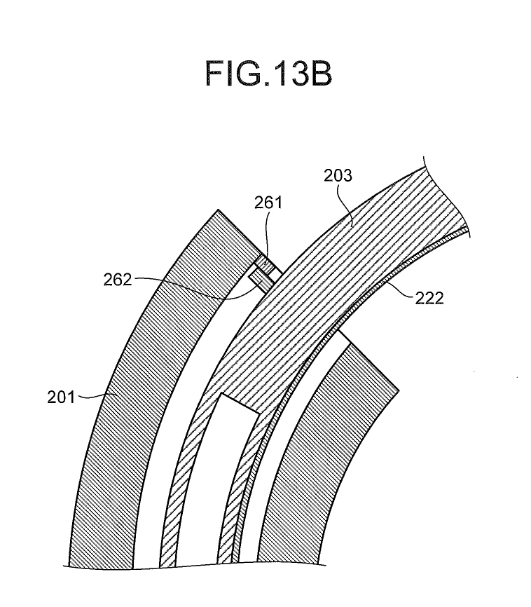

FIG. 13A is a schematic diagram illustrating a configuration of a guidance device according to Modified Example 2-3. As illustrated in FIG. 13A, the guidance device according to Modified Example 2-3 has a configuration in which stoppers 261 and 262 for restricting the movement of the lid portion 203 is further provided in the guidance device illustrated in FIG. 12.

FIG. 13B is a partial enlarged view of the stoppers 261 and 262 illustrated in FIG. 13A. The stopper 261 is provided on the inner circumferential surface of the outer wall 214 of the curved portion 211 and the stopper 262 is provided on the outer circumferential surface of the lid portion 203. These stoppers 261 and 262 are provided at a position at which a small gap is formed between the distal end of the lid portion 203 and the planar portion 212 when the stopper 262 stops moving by being caught at the stopper 261. This gap is preferably set so as not to have an adverse effect on the efficiency of shielding the magnetic field M generated by the permanent magnet 110.

When such stoppers 261 and 262 are provided, it is possible to prevent the occurrence of a situation in which an operator has his or her fingers between the lid portion 203 and the planar portion 212 even when the lid portion 203 abruptly moves to the shielding position due to the stopped electric power supply.

### Modified Example 2-4

In the guidance device according to the second embodiment, an energy storage unit such as a battery may be provided in the motor 236 similarly to Modified Example 1-3. In this case, electrical energy necessary for at least moving the permanent magnet from the height h₄ to the height h₃ (see FIG. 10A and FIG. 10B) is stored in the battery. In this way, when the electric power supply from the power source unit 105 to the motor 236 stops abruptly, electric power is supplied from the battery to the motor 236 and the permanent magnet is moved at a controlled speed from the height h₄ to the height h₃ by the driving force of the motor 236. In line with this movement, the lid portion 203 also moves to the shielding position at a speed corresponding to the moving speed of the permanent magnet 110.

### Third Embodiment

Next, the third embodiment of the present invention will be described.

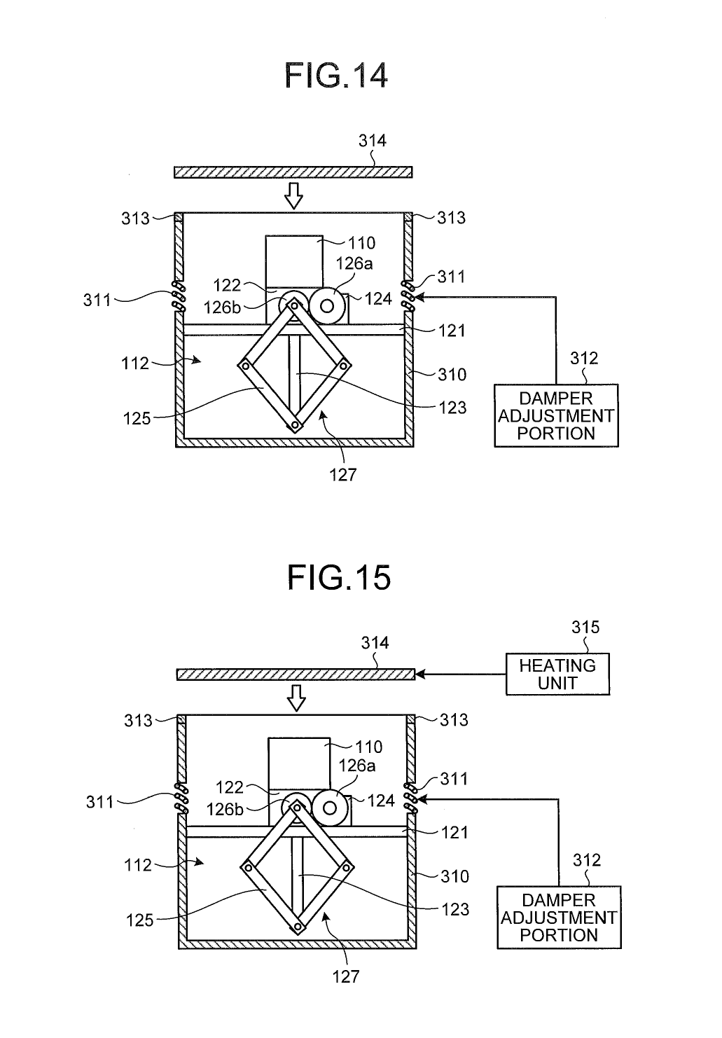

FIG. 14 is a schematic diagram illustrating a configuration of an accommodating portion of a permanent magnet used in a guidance device according to the third embodiment of the present invention. The guidance device according to the third embodiment has a configuration in which an accommodating portion 310 illustrated in FIG. 14 is provided instead of the accommodating portion 111 in the guidance device 100 illustrated in FIGS. 1A and 1B. The accommodating portion 310 enables the magnetic field M generated by the permanent magnet 110 to be shielded during maintenance of the guidance device.

The accommodating portion 310 is a box-shaped container of which the upper end is open and is formed of a ferromagnetic substance such as iron. A damper 311 is formed in a portion of a side wall of the accommodating portion 310. A damper adjustment portion 312 for adjusting the length of the damper 311 is provided in the damper 311 and the length of the damper 311 can be adjusted by electrical control. Moreover, an impact absorbing member 313 formed of an elastic member such as rubber, for example, is provided on an upper end surface of the side wall of the accommodating portion 310.

The permanent magnet 110 supported by the magnet holding mechanism 112 is disposed in the accommodating portion 310. Although the magnet holding mechanism 112 has the same configuration as the first embodiment, the lid portions 113 and 114 that cover the opening of the accommodating portion 310 and the wires 128a and 128b are removed (see FIG. 3A and FIG. 3B).

During maintenance, a maintenance magnetic field shielding portion 314 is used instead of the lid portions 113 and 114. The maintenance magnetic field shielding portion 314 is a planar member formed of a ferromagnetic substance such as iron, for example, and can shield the magnetic field M generated by the permanent magnet 110 more effectively than the lid portions 113 and 114 used during the examination based on the capsule endoscope 10.

Next, a maintenance method of the guidance device according to the third embodiment will be described. First, the length of the damper 311 is extended to the longest by the damper adjustment portion 312. Subsequently, the maintenance magnetic field shielding portion 314 is mounted on the upper end surface of the accommodating portion 310 to seal the opening. Here, the reason why the length of the damper 311 is extended to the longest is to minimize the magnetic attracting force of the permanent magnet 110 acting on the maintenance magnetic field shielding portion 314 when the maintenance magnetic field shielding portion 314 is mounted. Since the impact absorbing member 313 is provided at the end surface of the side wall of the accommodating portion 310, it is possible to absorb the impact when the maintenance magnetic field shielding portion 314 is mounted on the accommodating portion 310.

Subsequently the damper 311 is adjusted to a desired length by the damper adjustment portion 312. In this case, since the gravity and the magnetic attracting force of the permanent magnet 110 act on the maintenance magnetic field shielding portion 314, the length is decreased while controlling the speed of displacement of the damper 311. After that, maintenance of the guidance device is performed.

After the maintenance ends, the length of the damper 311 is extended to the longest while controlling the speed of displacement of the damper 311. This is to minimize the magnetic attracting force acting on the maintenance magnetic field shielding portion 314. Further, the maintenance magnetic field shielding portion 314 is removed and the lid portions 113 and 114 (see FIG. 3A and FIG. 3B) are attached instead and the lid portions 113 and 114 and the magnet holding mechanism 112 are connected by the wires 128a and 128b. In this way, a series of maintenance operations ends.

As described above, according to the third embodiment, by using the maintenance magnetic field shielding portion 314, the maintenance of the guidance device can be performed more safely. Moreover, in this case, since the maintenance magnetic-field shielding portion 314 is attached and detached in a state in which the length of the damper 311 is extend to the longest so that the magnetic attracting force acting on the maintenance magnetic field shielding portion 314 is minimized, the attachment and detachment operation can be performed more safely.

### Modified Example 3-1

Next, Modified Example 3-1 of the third embodiment of the present invention will be described.

FIG. 15 is a schematic diagram illustrating a configuration of a maintenance accommodating portion used in a guidance device according to Modified Example 3-1. As illustrated in FIG. 15, the accommodating portion of Modified Example 3-1 further includes a heating unit 315 for heating the maintenance magnetic field shielding portion 314 in the configuration illustrated in FIG. 14.

Next, a maintenance method of the guidance device according to Modified Example 3-1 will be described. First, the length of the damper 311 is extended to the longest by the damper adjustment portion 312. Subsequently, the maintenance magnetic field shielding portion 314 is mounted on the upper end surface of the accommodating portion 310 to seal the opening.

Subsequently, the maintenance magnetic field shielding portion 314 is heated up to the Curie temperature by the heating unit 315 to demagnetize the maintenance magnetic field shielding portion 314. After that, the heating unit 315 stops heating.

Subsequently the damper 311 is adjusted to a desired length by the damper adjustment portion 312. In this case, since the maintenance magnetic field shielding portion 314 is demagnetized and the magnetic attracting force of the permanent magnet 110 does not act on the maintenance magnetic field shielding portion 314, the height of the damper 311 can be adjusted more easily than the third embodiment. After that, when the temperature of the maintenance magnetic field shielding portion 314 decreases to a safe temperature, the maintenance of the guidance device is performed. The maintenance magnetic field shielding portion 314 may be cooled using a cooling device or the like and may be cooled naturally. Moreover, the maintenance magnetic field shielding portion 314 may be covered with a heat insulating material so that the user does not get burned during the maintenance.

After the maintenance ends, the maintenance magnetic field shielding portion 314 is heated by the heating unit 315 to demagnetize the maintenance magnetic field shielding portion 314 again. In this state, the length of the damper 311 is extended to the longest. Further, when the temperature of the maintenance magnetic field shielding portion 314 decreases to a safe temperature, the maintenance magnetic field shielding portion 314 is removed, the lid portions 113 and 114 (see FIG. 3A and FIG. 3B) are attached instead, and the lid portions 113 and 114 and the magnet holding mechanism 112 are connected by the wires 128a and 128b. In this way, a series of maintenance operations ends.

As described above, according to Modified Example 3-1, since the length of the damper 311 is adjusted after the maintenance magnetic field shielding portion 314 is demagnetized, it is possible to suppress the influence of the magnetic attracting force of the permanent magnet 110, on the maintenance magnetic field shielding portion 314 and to easily control the speed of displacement of the damper 311. Thus, the length of the damper 311 can be adjusted with the minimum necessary driving force.

### Fourth Embodiment

Next, the fourth embodiment of the present invention will be described.

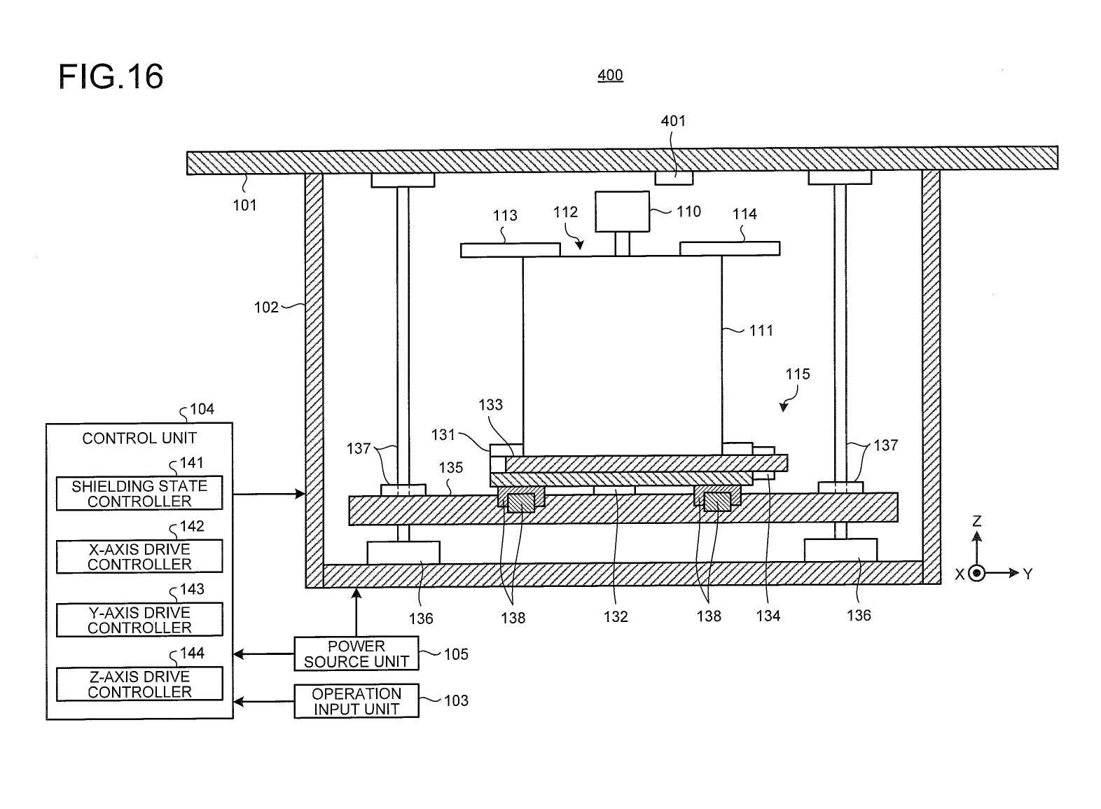

FIG. 16 is a schematic diagram illustrating a configuration of a guidance device according to the fourth embodiment of the present invention. As illustrated in FIG. 16, a guidance device 400 according to the fourth embodiment has a configuration in which a magnetic field display portion 401 is further added to the guidance device 100 illustrated in FIGS. 1A and 1B. The respective constituent elements of the guidance device 400 other than the magnetic field display portion 401 have the same configuration as those of the first embodiment. Moreover, FIG. 16 illustrates a state in which the guidance device 400 is in the non-shielding state.

The magnetic field display portion 401 is provided near the bed 101 and the accommodating portion 111 that accommodates the permanent magnet 110 and at a position at which the magnetic field display portion 401 is visible from the outside of the guidance device 400. The magnetic field display portion 401 displays the state of magnetic field at the position of the magnetic field display portion 401.

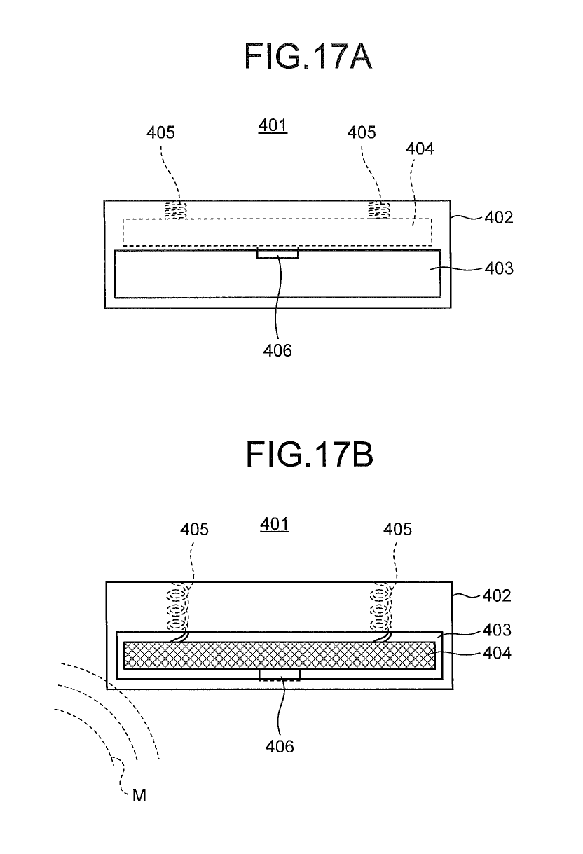

FIGS. 17A and 17B are schematic diagrams illustrating the magnetic field display portion 401 at an enlarged scale. Among these drawings, FIG. 17A illustrates the state of the magnetic field display portion 401 when the guidance device 400 is in the shielding state and FIG. 17B illustrates the state of the magnetic field display portion 401 when the guidance device 400 is in the non-shielding state.

The magnetic field display portion 401 includes a casing 402, a transparent window 403 formed on a side wall of the casing 402, a color bar 404 provided inside the casing 402, springs 405 that connect the color bar 404 to the inner wall of the casing 402, and a magnetic member 406 fixed to a lower portion of the color bar 404. The transparent window 403 is formed by forming a through-hole in the side wall of the casing 402 and fitting a transparent member such as a transparent plastic into the through-hole.

The color bar 404 is a planar member colored in a generally highly visible color such as red or orange and is suspended from the ceiling of the casing 402 with the springs 405 interposed. In the fourth embodiment, the color bar 404 corresponds to a display unit that indicates the non-shielding state when the color bar 404 is visible from the transparent window 403. In FIG. 17B, the colored portion is hatched. The magnetic member 406 is a member formed of a magnetic substance such as an iron plate or a small piece of iron.

When the guidance device 400 is in the shielding state, the magnetic member 406 is not influenced by the magnetic field generated by the permanent magnet 110 inside the accommodating portion 111. Thus, as illustrated in FIG. 17A, the color bar 404 and the magnetic member 406 are pulled upward (to the non-display position) inside the casing 402 by the elastic force of the springs 405 and the color bar 404 is invisible from the outside of the transparent window 403.

When the guidance device 400 enters the non-shielding state (see FIG. 16), the magnetic member 406 receives a downward force due to the magnetic attracting force of the magnetic field M generated by the permanent magnet 110. In this way, as illustrated in FIG. 17B, the color bar 404 is also pulled downward to move to the position (the display position) facing the transparent window 403. As a result, the color bar 404 is visible from the outside of the transparent window 403.

When the guidance device 400 enters the shielding state again, the magnetic member 406 is released from the magnetic attracting force of the magnetic field M generated by the permanent magnet 110. In this way, as illustrated in FIG. 17A, the color bar 404 and the magnetic member 406 are pulled upward by the elastic force of the springs 405 and the color bar 404 is invisible from the outside of the transparent window 403.

As described above, according to the fourth embodiment, the user can easily understand whether the guidance device 400 is in the shielding state or the non-shielding state by checking the transparent window 403 of the magnetic field display portion 401. Thus, for example, when the permanent magnet 110 is not completely accommodated in the accommodating portion 111 although the guidance device 400 is not used, the user can immediately perceive the unintended leakage of the magnetic field M.

According to the fourth embodiment, since the magnetic field display portion 401 is formed without using an electrical structure, even when an unexpected power failure or the like occurs, the user can understand the state (the shielding state or the non-shielding state) of the guidance device 400 accurately. Moreover, since the magnetic field display portion 401 works without using any power, the guidance device is inexpensive and is highly safe.

### Modified Example 4-1

Next, Modified Example 4-1 of the fourth embodiment of the present invention will be described.

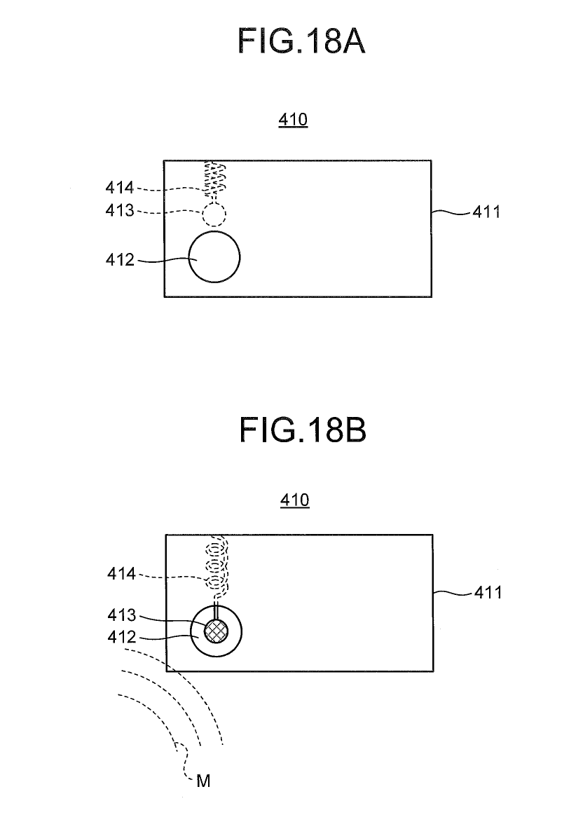

FIGS. 18A and 18B are schematic diagrams illustrating a magnetic field display portion included in a guidance device according to Modified Example 4-1. The guidance device according to Modified Example 4-1 includes a magnetic field display portion 410 illustrated in FIGS. 18A and 18B instead of the magnetic field display portion 401 illustrated in FIG. 16. Among these drawings, FIG. 18A illustrates the magnetic field display portion 410 when the guidance device is in the shielding state and FIG. 18B illustrates the magnetic field display portion 410 when the guidance device is in the non-shielding state. The respective constituent elements of the guidance device according to Modified Example 4-1 other than the magnetic field display portion 410 have the same configuration as those of the first embodiment.

The magnetic field display portion 410 includes a casing 411, a transparent window 412 formed on a side wall of the casing 411, a color ball 413 provided inside the casing 411, and a spring 414 that connects the color ball 413 to an inner wall of the casing 411. The transparent window 412 is formed by forming a through-hole in the side wall of the casing 411 and fitting a transparent member such as a transparent plastic into the through-hole.

The color ball 413 is a spherical member formed of a magnetic substance such as iron, of which the surface is colored in a generally highly visible color such as red or orange and is suspended from the ceiling of the casing 411 with the spring 414 interposed. In Modified Example 4-1, the color ball 413 corresponds to a display unit that indicates the non-shielding state when the color ball 413 is visible from the transparent window 412. In FIG. 18B, the colored portion is hatched.

When the guidance device is in the shielding state, the color ball 413 is not influenced by the magnetic field generated by the permanent magnet 110. Thus, as illustrated in FIG. 18A, the color ball 413 is pulled upward (to the non-display position) inside the casing 411 by the elastic force of the spring 414 and the color ball 413 is invisible from the outside of the transparent window 412.

When the guidance device enters the non-shielding state (see FIG. 16), the color ball 413 receives a downward force due to the magnetic attracting force of the magnetic field M generated by the permanent magnet 110 and moves to the position (the display position) facing the transparent window 412 as illustrated in FIG. 18B. As a result, the color ball 413 is visible from the outside of the transparent window 412.

When the guidance device enters the shielding state again, the color ball 413 is released from the magnetic attracting force of the magnetic field M generated by the permanent magnet 110. In this way, as illustrated in FIG. 18A, the color ball 413 is pulled upward by the elastic force of the spring 414 and the color ball 413 is invisible from the outside of the transparent window 412.

### Modified Example 4-2

Next, Modified Example 4-2 of the fourth embodiment of the present invention will be described.

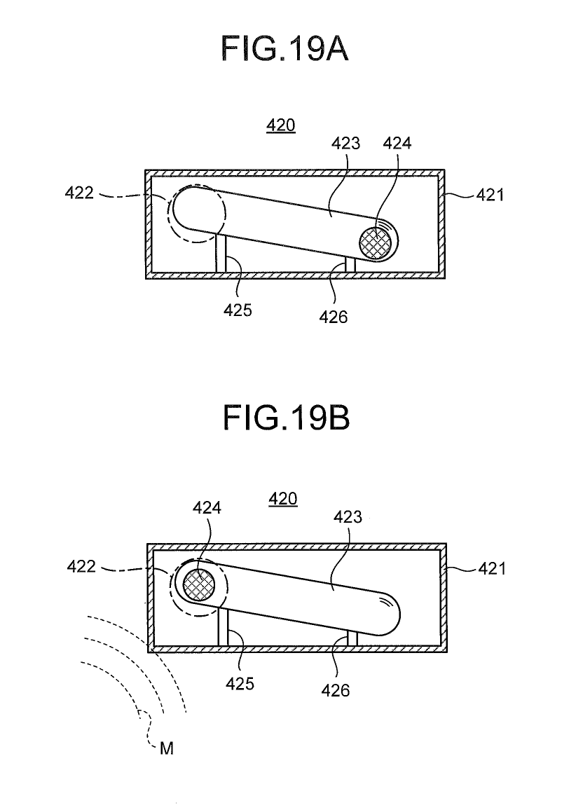

FIGS. 19A and 19B are schematic diagrams illustrating an inner configuration of a magnetic field display portion included in a guidance device according to Modified Example 4-2. The guidance device according to Modified Example 4-2 includes a magnetic field display portion 420 illustrated in FIGS. 19A and 19B instead of the magnetic field display portion 401 illustrated in FIG. 16. Among these drawings, FIG. 19A illustrates the magnetic field display portion 420 when the guidance device is in the shielding state and FIG. 19B illustrates the magnetic field display portion 420 when the guidance device is in the non-shielding state. The respective constituent elements of the guidance device according to Modified Example 4-2 other than the magnetic field display portion 420 have the same configuration as those of the first embodiment.

The magnetic field display portion 420 includes a casing 421, a transparent window 422 formed on a side wall of the casing 421, a tubular member 423 provided inside the casing 421, and a color ball 424 disposed inside the tubular member 423.

The tubular member 423 is formed of a transparent member like a transparent plastic, of which the inner state can be visible from the outside and is disposed so that the position of one end is higher than that of the other end with the aid of supporting members 425 and 426. The transparent window 422 is formed by forming a through-hole in the side wall of the casing 421 and fitting a transparent member such as a transparent plastic into the through-hole. The transparent window 422 is provided at a position such that the end on which the position of the tubular member 423 is higher is visible from the outside of the casing 421.

The color ball 424 is a spherical member in which a magnetic substance such as iron is provided in at least a portion thereof, and of which the surface is colored in a generally highly visible color such as red or orange, and is disposed so as to be movable inside the tubular member 423. In Modified Example 4-2, the color ball 424 corresponds to a display unit that indicates the non-shielding state when the color ball 424 is visible from the transparent window 422. In FIG. 19B, the colored portion is hatched in a lattice form.

When the guidance device is in the shielding state, the color ball 424 is not influenced by the magnetic field generated by the permanent magnet 110. Thus, as illustrated in FIG. 19A, the color ball 424 remains at the end (the non-display position) on which the position of the tubular member 423 is lower due to the effect of the gravity and the color ball 424 is invisible from the outside of the transparent window 422.

When the guidance device enters the non-shielding state (see FIG. 16), the color ball 424 receives a leftward force in the drawing due to the magnetic attracting force of the magnetic field M generated by the permanent magnet 110. In this way, as illustrated in FIG. 19B, the color ball 424 moves leftward along the slope surface of the tubular member 423 and remains at the end (the display position) on which the position is higher. In this case, the color ball 424 is visible from the outside of the transparent window 422.

When the guidance device enters the shielding state again, the color ball 424 is released from the magnetic attracting force of the magnetic field M generated by the permanent magnet 110, and moves rightward in the drawing along the slope surface of the tubular member 423 by the effect of the gravity. In this way, as illustrated in FIG. 19A, the color ball 424 is invisible from the outside of the transparent window 422.

### Modified Example 4-3

Next, Modified Example 4-3 of the fourth embodiment of the present invention will be described.

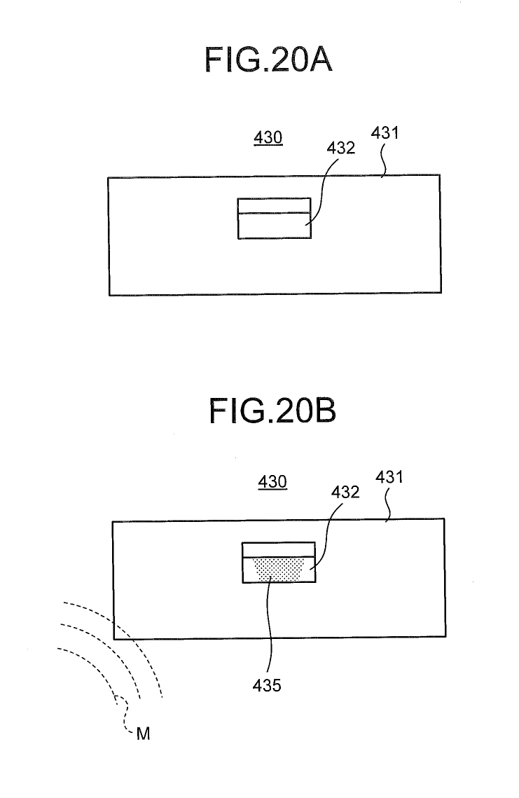

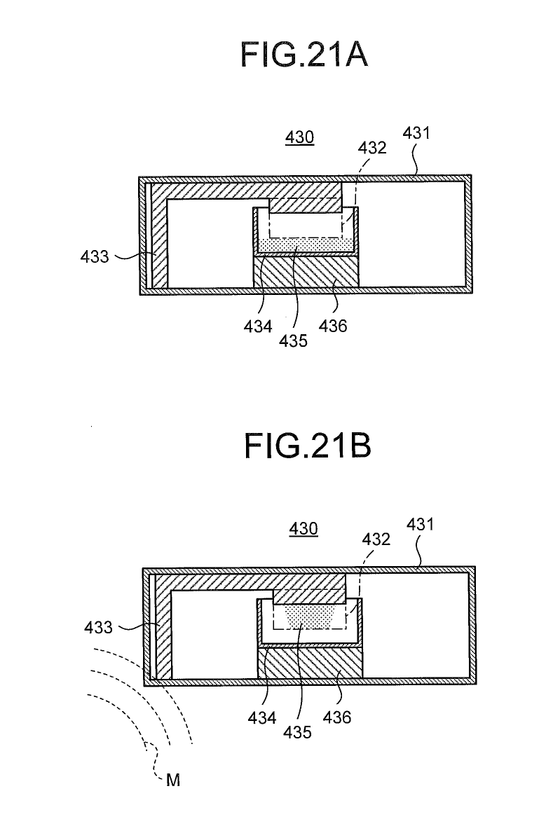

FIGS. 20A and 20B are schematic diagrams illustrating an appearance of a magnetic field display portion included in a guidance device according to Modified Example 4-3. Moreover, FIGS. 21A and 21B are schematic diagrams illustrating an inner configuration of the magnetic field display portion. The guidance device according to Modified Example 4-3 includes a magnetic field display portion 430 illustrated in FIG. 20A to FIG. 21B instead of the magnetic field display portion 401 illustrated in FIG. 16. Among these drawings, FIGS. 20A and 21A illustrate the magnetic field display portion 430 when the guidance device is in the shielding state and FIGS. 20B and 21B illustrate the magnetic field display portion 430 when the guidance device is in the non-shielding state. The respective constituent elements of the guidance device according to Modified Example 4-3 other than the magnetic field display portion 430 have the same configuration as those of the first embodiment.

The magnetic field display portion 430 includes a casing 431, a transparent window 432 formed on a side wall of the casing 431, a magnetic field reacting portion 433 provided inside the casing 431, a container 434 provided inside the casing 431, and a display member 435 accommodated in the container 434. The transparent window 432 is formed by forming a through-hole in the side wall of the casing 431 and fitting a transparent member such as a transparent plastic into the through-hole.

The magnetic field reacting portion 433 is formed of a ferromagnetic substance like iron which is easily magnetized by the influence of a surrounding magnetic field. The magnetic field reacting portion 433 has an approximately L-shape, for example, and is disposed so that one side of the L-shape follows an inner wall of the casing 431 close to the permanent magnet 110 (see FIG. 16).

The container 434 is formed of a transparent member like a transparent plastic, of which the inner state can be visible from the outside, for example, and is fixed at a predetermined position inside the casing 431 by a supporting member 436. The display member 435 is a member in which a magnetic substance having a granular (powder-like) form such as iron sand, for example, is colored in a generally highly visible color such as red or orange and is disposed up to a predetermined height inside the container 434. The transparent window 432 is provided near the container 434 at a position higher than the height of the display member 435 disposed in the container 434. In Modified Example 4-3, the display member 435 corresponds to a display unit that indicates the non-shielding state when the display member 435 is visible from the transparent window 432.

When the guidance device is in the shielding state, the magnetic field reacting portion 433 is not influenced by the magnetic field generated by the permanent magnet 110. Thus, as illustrated in FIG. 21A, the display member 435 remains at the bottom (the non-display position) in the container 434 and the display member 435 is invisible from the outside of the transparent window 432 as illustrated in FIG. 20A.

When the guidance device enters the non-shielding state (see FIG. 16), the magnetic field reacting portion 433 is magnetized by the magnetic field M generated by the permanent magnet 110. In this way, as illustrated in FIG. 21B, the display member 435 in the container 434 is attracted to the end (the display position) of the magnetic field reacting portion 433, and the display member 435 is visible from the outside of the transparent window 432 as illustrated in FIG. 20B.

When the guidance device enters the shielding state again, the magnetic field reacting portion 433 is demagnetized and the display member 435 is released from the magnetic attracting force of the magnetic field reacting portion 433 to gather at the bottom of the container 434 as illustrated in FIG. 21A. In this way, as illustrated in FIG. 20A, the display member 435 is invisible from the outside of the transparent window 432.

The surface of the magnetic field reacting portion 433 may be coated with a low-friction material so that the display member 435 is reliably separated from the magnetic field reacting portion 433 when the guidance device transitions from the non-shielding state to the shielding state. Moreover, a colored fluid having magnetism may be used as the display member 435.

### Modified Example 4-4

Next, Modified Example 4-4 of the fourth embodiment of the present invention will be described.

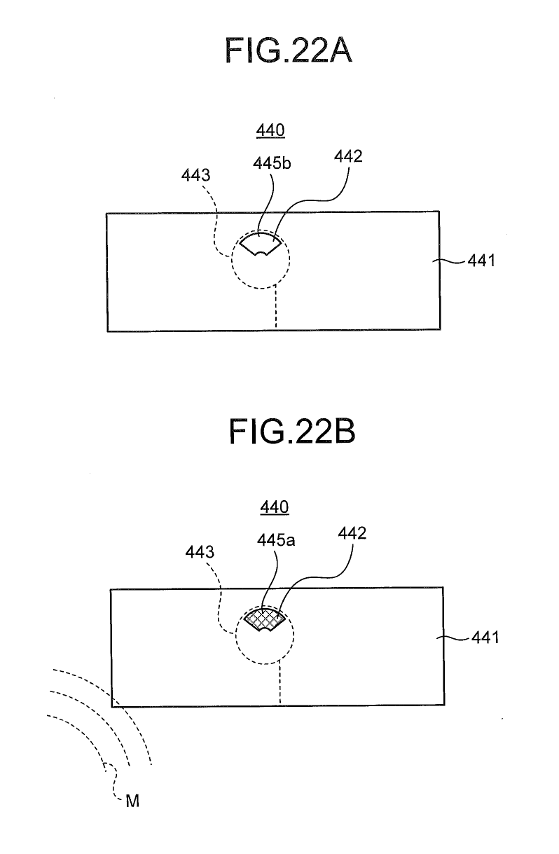

FIGS. 22A and 22B are schematic diagrams illustrating an appearance of a magnetic field display portion included in a guidance device according to Modified Example 4-4. The guidance device according to Modified Example 4-4 includes a magnetic field display portion 440 illustrated in FIGS. 22A and 22B instead of the magnetic field display portion 401 illustrated in FIG. 16. Among these drawings, FIG. 22A illustrates the magnetic field display portion 440 when the guidance device is in the shielding state and FIG. 22B illustrates the magnetic field display portion 440 when the guidance device is in the non-shielding state. The respective constituent elements of the guidance device according to Modified Example 4-4 other than the magnetic field display portion 440 have the same configuration as those of the first embodiment.

The magnetic field display portion 440 includes a casing 441, a transparent window 442 formed on a side wall of the casing 441, and a disk 443 rotatably provided inside the casing 441. The transparent window 442 is formed by forming a through-hole in the side wall of the casing 441 and fitting a transparent member such as a transparent plastic into the through-hole. Moreover, the disk 443 is disposed so that a portion of one surface thereof faces the transparent window 442.

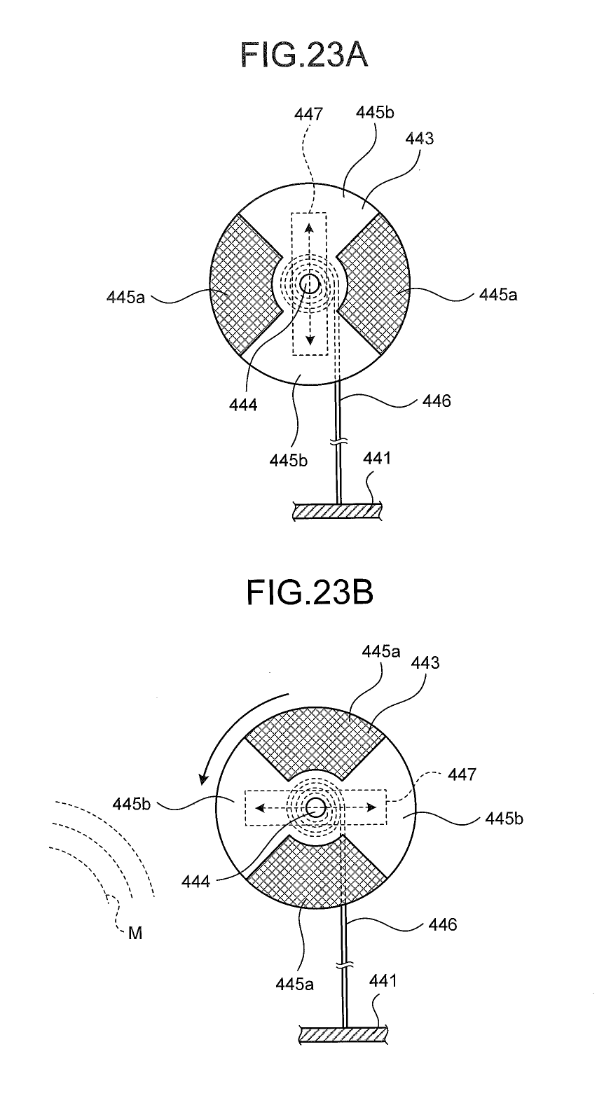

FIGS. 23A and 23B are schematic diagrams illustrating a configuration of the disk 443. Among these drawings, FIG. 23A illustrates the disk 443 when the guidance device is in the shielding state and FIG. 23B illustrates the disk 443 when the guidance device is in the non-shielding state. A rotating shaft member 444, a spiral spring 446 wound around the rotating shaft member 444, and a permanent magnet 447 fixed to a rear surface of the disk 443 are provided in the disk 443.

The disk 443 is attached to the casing 441 so as to be rotatable with the aid of the rotating shaft member 444 provided at the center of rotation. Moreover, a partial area of a surface (hereinafter referred to as a front surface) of the disk 443 facing the transparent window 442 is colored in a generally highly visible color such as red or orange. Hereinafter, the area of the front surface of the disk 443, colored in the highly visible color will be referred to as a coated area 445a and an area that is not colored will be referred to as a non-coated area 445b. In Modified Example 4-4, the coated area 445a corresponds to a display unit that indicates the non-shielding state when the coated area 445a is visible from the transparent window 442. In FIGS. 22B to 23B, the colored portion is hatched in a lattice form.

One end of the spiral spring 446 is fixed to an outer circumferential surface of the rotating shaft member 444 and the other end is fixed to an inner bottom surface of the casing 441. Moreover, the permanent magnet 447 has a rod shape and is fixed to a position that passes through the center of rotation of the disk 443. The permanent magnet 447 is provided so that the magnetization direction thereof is aligned in a vertical direction when the spiral spring 446 is in a natural state. In FIGS. 23A and 23B, a two-way arrow depicted on the permanent magnet 447 indicates the magnetization direction of the permanent magnet 447.

When the guidance device is in the shielding state, the permanent magnet 447 is not influenced by the magnetic field generated by the permanent magnet 110 in the accommodating portion 111. Thus, as illustrated in FIG. 23A, the disk 443 is maintained in a direction in which the magnetization direction of the permanent magnet 447 is aligned in the vertical direction. In this case, the coated area 445a is disposed at a position (the non-display position) concealed from the transparent window 442 and the coated area 445a is invisible from the outside of the transparent window 442 as illustrated in FIG. 22A.

When the guidance device enters the non-shielding state (see FIG. 16), torque is generated in the permanent magnet 447 in a direction indicated by an arrow illustrated in FIG. 23B by the influence of the magnetic field M generated by the permanent magnet 110 and the permanent magnet 447 and the disk 443 rotate. In this way, as illustrated in FIG. 22B, the coated area 445a moves to a position (the display position) facing the transparent window 442 and the coated area 445a is visible from the outside of the transparent window 442.

When the guidance device enters the shielding state again, the permanent magnet 447 is released from the influence of the magnetic field M generated by the permanent magnet 110, and the disk 443 and the permanent magnet 447 rotate in the direction opposite to the arrow in FIG. 23B by the restoring force of the spiral spring 446. In this way, as illustrated in FIG. 23A, the magnetization direction of the permanent magnet 447 is aligned again in the vertical direction. In this case, as illustrated in FIG. 22A, the coated area 445a is invisible from the outside of the transparent window 442.

According to some embodiments, the shielding member moving mechanism is provided to move the shielding member between the shielding position and the non-shielding position in conjunction with the movement of the second permanent magnet in the vertical direction. Thus, when the electric power supply to the driving unit stops, the second permanent magnet moves to the lowest position in the movable range and the shielding member moves to the shielding position in conjunction with the movement of the second permanent magnet. Thus, it is possible to immediately and reliably shield a magnetic field generated by the second permanent magnet to be confined in the accommodating portion even when an unsuspected situation occurs on performing the guidance of the capsule endoscope is performed inside a subject.

The first and fourth embodiments described above and Modified Examples thereof are only examples for implementing the present invention, and the present invention is not limited to these examples. Moreover, various inventions can be invented by appropriately combining a plurality of constituent elements disclosed in the embodiments and modified examples of the present invention. The present invention can be variously modified depending on specifications and the like. Furthermore, it is obvious from the above description that other various embodiments are possible within the scope of the present invention.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A guidance device for guiding a capsule medical device having a magnet included therein by allowing a magnetic field to act on the magnet, comprising:
a permanent magnet provided outside the capsule medical device, the permanent magnet generating a magnetic field that acts on the magnet;
an accommodating portion configured to accommodate the permanent magnet;
a shielding member configured to shield the magnetic field generated by the permanent magnet accommodated in the accommodating portion;
a supporting mechanism configured to support the permanent magnet to be movable in a vertical direction, the supporting mechanism being configured to support the permanent magnet between a first position at which the permanent magnet is accommodated in the accommodating portion and a second position at which the capsule medical device can be guided by the magnetic field generated by the permanent magnet;
a driving unit configured to operate the supporting mechanism to move the permanent magnet along a vertical direction upon receiving an electric power supply; and
a shielding member moving mechanism configured to move the shielding member between a shielding position at which the magnetic field generated by the permanent magnet is confined in the accommodating portion and a non-shielding position at which the magnetic field generated by the permanent magnet is not confined in the accommodating portion in conjunction with an operation of the supporting mechanism causing the permanent magnet to move in the vertical direction, wherein
when the electric power supply to the driving unit stops, the shielding member moving mechanism moves the shielding member to the shielding position in conjunction with an operation of the supporting mechanism causing the permanent magnet to move to the first position.

2. The guidance device according to claim 1, wherein
the driving unit is configured to operate the supporting mechanism to maintain the permanent magnet at the second position against the gravity upon receiving the electric power supply,
the shielding member moving mechanism is configured to maintain the shielding member at the non-shielding position while the permanent magnet is at the second position, and
when the electric power supply to the driving unit stops, the permanent magnet falls to the first position due to its own weight.

3. The guidance device according to claim 1, further comprising:
an energy storage unit configured to store necessary energy for at least moving the permanent magnet from the second position to the first position, the energy being supplied to the driving unit, wherein
the driving unit is configured to maintain the permanent magnet at the second position against the gravity upon receiving the electric power supply,
the shielding member moving mechanism is configured to maintain the shielding member at the non-shielding position while the permanent magnet is at the second position, and
when the electric power supply to the driving unit stops, the energy storage unit supplies the stored energy to the driving unit and the supporting mechanism moves the permanent magnet from the second position to the first position.

4. The guidance device according to claim 1, wherein
the shielding member has a cross-section having a circular arc shape and has a curved shape that is protruded toward an outside of the accommodating portion, and
the shielding member moving mechanism is configured to move the shielding member along a circular arc-shaped trajectory having the same curvature as the circular arc shape.

5. The guidance device according to claim 1, wherein
the shielding member is formed of a ferromagnetic substance.

6. The guidance device according to claim 1, wherein
the accommodating portion is formed of a ferromagnetic substance.

7. The guidance device according to claim 1, wherein
the first position is a lowest position in a movable range of the permanent magnet.

8. The guidance device according to claim 1, further comprising:
a magnetic field display portion provided outside the accommodating portion and configured to display whether the magnetic field generated by the permanent magnet is confined in the accommodating portion or not, wherein
the magnetic field display portion includes:
a casing in which a window through which an inside of the casing is configured to be observed visually from an outside of the casing is formed in a portion of a wall surface; and
a display unit which is provided in the casing to be movable between a non-display position at which the inside of the casing cannot be observed visually from the outside of the casing and a display position at which the inside of the casing can be observed visually from the outside of the casing through the window, and
the display unit is configured to move from the non-display position to the display position with the magnetic field generated by the permanent magnet.

9. A capsule medical device guidance system comprising:
the guidance device according to claim 1; and
the capsule medical device.
